(19) United States
(12) Patent Application Publication
(10) Pub. No.: US 2020/0340062 A1
(43) Pub. Date: Oct. 29, 2020

(54) PROGNOSTIC MARKERS FOR CANCER RECURRENCE

(71) Applicant: University of Southern California, Los Angeles, CA (US)

(72) Inventor: Bodour Salhia, Los Angeles, CA (US)

(21) Appl. No.: 16/639,065

(22) PCT Filed: Aug. 18, 2018
(86) PCT No.: PCT/IB2018/056255

§ 371 Date: Feb. 13, 2020

Related U.S. Application Data
(60) Provisional application No. 62/547,732, filed on Aug. 18, 2017.

(51) Int. Cl. C12Q 1/6886

(52) U.S. Cl. CPC C12Q 1/6886; C12Q 2600/112; C12Q 2600/154; C12Q 2600/118

(57) ABSTRACT

There is a need to accurately monitor a cancer patient's risk status after completion of therapy due to residual disease. Herein provided are methods related to detection of cancer and cancer recurrence in a subject using detection of cell-free DNA methylation.

### CROSS-REFERENCE TO RELATED APPLICATIONS

This application is a U.S. National Stage Application under 35 U.S.C. § 371 of International Application No. PCT/IB2018/056255, filed Aug. 18, 2018, which claims the benefit under 35 U.S.C. § 119(e) of U.S. Provisional Application No. 62/547,732, filed August 18, 2017, the contents each of which are incorporated by reference into the present disclosure.

### BACKGROUND

Despite improvements in breast cancer screening, diagnosis, and treatment, there are patients who develop metastasis and succumb to their disease. Once patients develop metastatic breast cancer (MBC), their disease is treatable but not curable, and the 5-year survival for patients with MBC remains below 25%. The ability to predict which patients will develop distant disease recurrence is still based on relatively crude factors. A number of clinico-pathological criteria have been established as breast cancer prognostic markers, which are used to determine risk of recurrence and stratify patients into high and low risk groups. The risk of distant metastasis increases with bigger tumor size, the presence and number of lymph-node involvement, lack of estrogen receptor (ER) expression, over-expression of Her2, a high proliferative index, lymphovascular invasion, and higher histopathological differentiation (grade). Even with these clinico-pathologic criteria, clinicians are still unable to concretely define which groups of patients will be cured or will develop MBC regardless of whether they are stratified as having high-risk or low-risk disease.

Molecular profiles have improved our ability to determine the need of chemotherapy for those individuals who are deemed high-risk. However, no currently available profiles can precisely predict the clinical course of an individual and rely on the presence of tissue at a single time point. Therefore, clinicians are not able to accurately monitor a patient's risk status after completion of therapy due to residual disease. Described herein are methods useful in the pre-macrometastatic setting to indicate patients at a high risk of recurrence.

### SUMMARY OF THE DISCLOSURE

Applicant developed novel methods for molecular profiling of cancer that are useful for predicting or detecting cancer, cancer recurrence, and/or cancer metastasis. Thus, in one aspect, this disclosure provides a method for determining whether a subject is likely to have or develop cancer or cancer recurrence, the method comprising, or alternatively consisting essentially of, or yet further consisting of: (a) determining the level of DNA methylation at a genomic region within 10³ kb of at least one gene selected from RRAGC, RNF207, CAMTA1, IL17RE, Gp5, COX7B2, BANK1, LIMCH1, ANKRD33B, loc648987, HOXA2, NR_027387, ATG9B, KBTBD2, loc401321, MAFA, ANK1, SPAG1, PBX3, c9orf139, FUBP3, RABL6, DIP2C, CHFR, ZNF605, DZIP1, SLC35F4, ACSF2, ARHGAP23, FUZ, PBX4, UNC13A, ISM1, BMP2, loc286647, STAC2, TBX15, ESPN, PLEKHO1, c1orf95, HIVEp3, SPEG8, NR_038487, TANK, ARHGEF4, ZNF148, MIR548G, COX7B2, loc285548, Pi42Kb, PCDH7, FHDC1, GPR150, SLC6A3, VGLL2, NRN1, BLACE, WDR86, HOXA9, SOX17, ASS1, ALOX5, ZNF503, MAP6, EPS8L2, B4GALANT1, CLDN10, CLEC14A, PIF1, JPH3, SALL1, HIC1, ATP1B2, SRCIN1, NETO1, RCN3, and SEPT5-GP1BB in a sample isolated from the subject; (b) comparing the level of DNA methylation in the sample to the level of DNA methylation in a sample isolated from a cancer-free subject, a normal reference standard, or a normal reference cutoff value; and (c) determining that the subject is likely to have or develop cancer or cancer recurrence if the level of DNA methylation in the sample derived from the subject is greater than the level of DNA methylation in the sample isolated from a cancer-free subject, a normal reference standard, or a normal reference cutoff value. In some aspects, the DNA is cell-free DNA.

Also provided is a method for detecting the level of DNA methylation in a sample isolated from a subject suspected of having or developing cancer or early stage cancer, the method comprising, or alternatively consisting essentially of, or yet further consisting of determining the level of DNA methylation at a genomic region within 10³ kb of at least one gene selected from RRAGC, RNF207, CAMTA1, IL17RE, Gp5, COX7B2, BANK1, LIMCH1, ANKRD33B, loc648987, HOXA2, NR_027387, ATG9B, KBTBD2, loc401321, MAFA, ANK1, SPAG1, PBX3, c9orf139, FUBP3, RABL6, DIP2C, CHFR, ZNF605, DZIP1, SLC35F4, ACSF2, ARHGAP23, FUZ, PBX4, UNC13A, ISM1, BMP2, loc286647, STAC2, TBX15, ESPN, PLEKHO1, c1orf95, HIVEp3, SPEG8, NR_038487, TANK, ARHGEF4, ZNF148, MIR548G, COX7B2, loc285548, Pi42Kb, PCDH7, FHDC1, GPR150, SLC6A3, VGLL2, NRN1, BLACE, WDR86, HOXA9, SOX17, ASS1, ALOX5, ZNF503, MAP6, EPS8L2, B4GALANT1, CLDN10, CLEC14A, PIF1, JPH3, SALL1, HIC1, ATP1B2, SRCIN1, NETO1, RCN3, and SEPT5-GP1BB in the sample. In some aspects, the method further comprises comparing the measured level of DNA methylation in the sample to the level of DNA methylation in a sample isolated from a cancer free subject, a normal reference standard, or a normal reference cutoff value. In some aspects, the DNA is cell-free DNA.

In one aspect, the level of DNA methylation is determined at one or more CpG islands within 10³ kb of the selected gene or genes. In other aspects, the level of DNA methylation is determined at a genomic region within 900 kb, 800 kb, 700 kb, 600 kb, 500 kb, 400 kb, 300 kb, 200 kb, 100 kb, 50 kb, 10 kb, or 5 kb of the selected gene or genes.

In some aspects, the level of DNA methylation is determined at a genomic region within the selected gene or genes. Non-limiting examples include a genomic region within an untranslated region (UTR) of the selected gene or genes, a genomic region within 1.5 kb upstream of the transcription start site of the selected gene or genes, and a genomic region within the first exon of the selected gene or genes.

Also provided herein is a method for determining whether a subject is likely to have or develop cancer or early stage cancer, the method comprising, or alternatively consisting essentially of, or yet further consisting of: (a) determining the level of DNA methylation at one or more genomic regions selected from chr1:119,522,297-119,522,685, chr1:150,122,865-150,123,881, chr1:226,736,415-226,736,530, chr1:228,651,389-228,652,669, chr1:39,044,074-39,044,222, chr1:39,044,074-39,044,225, chr1:39,269,706-39,269,850, chr1:42,383,685-42,383,856, chr1:6,268,888-6,269,045, chr1:6,508,634-6,508,912, chr1:7,765,055-7,765,179, chr2:131,792,795-131,792,937, chr2:162,100,925-162,101,769, chr2:208,989,125-208,989,413, chr2:220,313,284-220,313,454, chr2:220,313,294-220,313,436, chr2:468,028-468,289, chr3:125,076,002-125,076,434, chr3:194,117,552-194,119,057, chr3:194,117,921-194,118,045, chr3:9,957,033-9,957,468, chr3:99,595,058-99,595,326, chr4:102,712,059-102,712,200, chr4:13,549,015-13,549,160, chr4:153,858,813-153,858,916, chr4:25,235,927-25,236,058, chr4:30,723,649-30,723,941, chr4:41,646,367-41,646,493, chr4:46,726,419-46,726,619, chr4:46,726,427-46,726,601, chr4:46,726,525-46,726,603, chr4:8,895,441-8,895,846, chr5:1,445,269-1,445,490, chr5:10,565,517-10,565,682, chr5:43,018,031-43,018,972, chr5:94,955,846-94,956,706, chr6:117,591,888-117,592,164, chr6:6,002,430-6,002,857, chr7:150,715,883-150,715,989, chr7:151,106,717-151,106,910, chr7:152,161,438-152,161,508, chr7:155,167,043-155,167,243, chr7:27,141,743-27,141,932, chr7:27,204,874-27,205,029, chr7:32,801,782-32,802,525, chr7:32,930,792-32,930,842, chr8:101,225,311-101,225,367, chr8:144,511,850-144,512,138, chr8:41,655,108-41,655,453, chr8:55,379,115-55,379,416, chr9:128,510,274-128,510,341, chr9:133,308,833-133,309,057, chr9:133,454,823-133,454,962, chr9:139,715,901-139,716,003, chr9:139,925,051-139,925,313, chr10:45,914,402-45,914,709, chr10:735,378-735,552, chr10:77,156,043-77,156,222, chr11:725,576-725,843, chr11:75,379,637-75,379,770, chr12:133,481,446-133,481,616, chr12:58,021,185-58,021,918, chr13:29,393,957-29,394,126, chr13:96,204,915-96,205,232, chr13:96,293,984-96,294,377, chr14:38,724,432-38,725,600, chr14:58,332,639-58,332,759, chr15:65,116,372-65,116,575, chr15:66,914,674-66,914,722, chr16:51,185,202-51,185,325, chr16:87,636,189-87,636,318, chr17:1,960,496-1,960,610, chr17:36,666,487-36,666,582, chr17:36,714,476-36,714,611, chr17:37,366,246-37,366,533, chr17:37,381,269-37,381,871, chr17:44,337,407-44,337,726, chr17:48,546,161-48,546,934, chr17:7,554,926-7,555,051, chr18:70,522,481-70,548,676, chr19:17,716,756-17,717,092, chr19:19,729,144-19,729,553, chr19:30,716,841-30,717,033, chr19:50,030,948-50,031,354, chr19:50,312,537-50,312,694, chr20:13,200,413-13,200,789, chr20:6,748,289-6,748,421, chr22:19,711,302-19,711,474, and chrX:130,929,860-130,930,244 in a sample isolated from the subject; (b) comparing the level of DNA methylation in the sample to the level of DNA methylation in a sample isolated from a cancer-free subject, a normal reference standard, or a normal reference cutoff value; and (c) determining that the subject is likely to have or develop cancer or cancer recurrence if the level of DNA methylation in the sample derived from the subject is greater than the level of DNA methylation in the sample isolated from a cancer-free subject, a normal reference standard, or a normal reference cutoff value. In some aspects, the DNA is cell-free DNA.

In some aspects, the DNA methylation level is determined with targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

In another aspect, the method further comprises performing one or more of targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

In some aspects, the sample isolated from the subject is a non-invasive or minimally invasive sample. Non-limiting examples include whole blood, plasma, serum, urine, feces, saliva, buccal mucosa, sweat, or tears. In a further aspect, the sample is cell-free and/or comprises cell-free DNA.

In some aspects, the methods determine whether a subject is likely to have or develop lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma, or recurrence or metastasis of lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma.

Also provided herein is a method for identifying screening, predictive, prognostic, or diagnostic markers for a disease, the method comprising, or alternatively consisting essentially of, or yet further consisting of: a) determining the methylation profile of a pool of cell free DNA samples isolated from subjects with the disease; b) determining the methylation profile of a pool of cell free DNA samples isolated from disease-free subjects or a normal reference standard; wherein each pool consists of equal amounts of cell free DNA; c) comparing the methylation profiles determined in a) and b); and d) selecting differentially methylated regions with greater than 40% differential value. In one aspect, the method further comprises validation of the selected regions. In some aspects, validation comprises targeted amplicon bisulfite sequencing.

### BRIEF DESCRIPTION OF THE DRAWINGS

FIGS. 1A-1C: Whole genome bisulfite sequencing (WGBS) reveals that metastatic breast cancer (MBC) methylation profiles differ from disease free survivors (DFS) and H, which are similar. FIG. 1A Heat scatterplots show % methylation values for pair-wise comparisons of three study groups. Numbers on the upper right corner denote Pearson correlation coefficients. The histograms on the diagonal are frequency of % methylation per cytosine for each pool. MBC demonstrates a shift to the left compared to the DFS and H, indicating genome-wide hypomethylation. FIG. 1B Hierarchical clustering of methylation profiles for each pool using Pearson's correlation distance and Ward's clustering method. FIG. 1C Principal Component Analysis of the methylation profiles of each cfDNA pool, showing PC1 and PC2 for each sample. Samples closer to each other in clustering or principal component space are similar in their methylation profiles

FIGS. 2A-2B: FIG. 2A Venn diagram showing the overlap of DML lists as generated by WGBS for H, DFS, and MBC sample comparisons. FIG. 2B Three pair-wise comparisons assessing cfDNA differential methylation between H, DFS, and MBC. Pie charts show percentages of differentially hyper- or hypomethylated CpG loci genome-wide and within the displayed genomic contexts. Greater than 90% of CpG loci are hypomethylated genome-wide in MBC compared with Healthy or DFS. The majority of hypermethylated loci in MBC occur within CpG islands. The number of DML and the percentages are shown within each pie chart.

FIGS. 3A-3B: FIG. 3A Circos plot graphing methylation state for each locus in the CpG island of 21 target genes. The hotspot region exists within each island. The inner circle (red) is MBC, middle circle is DFS (green), and outer circle is H (blue). Hypermethylation is evident in MBC for the target genes. FIG. 3B Vertical scatter plot showing all DML within target CPGIs for MBC versus DFS and H, respectively. Each point represents a CpG locus. Points plotted on the x-axis display the DMVs.

FIGS. 4A-4D: Comparison of WGBS to MiSeq (targeted amplicon sequencing). FIG. 4A Box plots representing percent methylation for DMLs in GPS, HTR1B, PCDH10, and UNC13A as called by both technologies. FIG. 4B Mean-Whisker plots displaying average methylation state of all amplicons assayed by MiSeq and WGBS. FIG. 4C Scatter plot of percent methylation value for the 36 CpGs assayed in H, DFS, and MBC by both MiSeq and WGBS. The correlation is reported as R²=0.768. FIG. 4D Pearson correlation coefficient for WGBS versus MiSeq for 36 CpGs assayed by targeted amplicon sequencing.

FIG. 5: Read coverage in DMLs of interest. Box plots show the depth of sequencing as determined by WGBS and MiSeq for 36 DMLs specific to GP5, HTR1B, PCDH10, and UNC13A in all pools of H (blue), DFS (green), and MBC (red). Coverage is shown as log 10.

FIG. 6: Patients with cancer present with different disease statuses as it relates to the degree of metastatic spread. Metastasis begins when malignant cells from the primary tumor acquire invasive phenotypes, penetrate the extra cellular matrix, and pass into the bloodstream. Circulating tumor cells (CTC) then travel through the bloodstream, adhere to the basement membrane, make a metastatic deposit and grow as a macrometastasis in their new site. There is a phase during the metastatic process where detection of micrometastatic cells may lead to prevention of macrometastatic lesions, which are incurable. (Adapted from A Perspective on Cancer Cell Metastasis; Chaffer and Weinberg. Science 25 Mar. 2011: vol. 331 no. 6024 1559-1564).

FIGS. 7A-7D: Analysis of 120 clinically annotated plasma samples for the Komen Tissue Bank representing 40 samples from Healthy individuals, 40 from disease free survivors (DFS) and 40 from patients with metastatic breast cancer (MBC). FIG. 7A Pie chart shows distribution of involved sites of distant metastases in the MBC group. FIG. 7B Vertical plot shows the number of years disease free in the DFS group. Two clusters are evident. FIG. 7C cfDNA extractions from 120 individual samples. Vertical scatterplot of DNA yield. Table is a summary of yield in nanograms. FIG. 7D Tapestation trace showing extraction of cfDNA at expected size (167 bp—middle peak).

FIGS. 8A-8B: WGBS reveals MBC methylation profiles differs from DFS and Healthy, which are similar. FIG. 8A Heat scatterplots show % methylation values for pair-wise comparisons of three study groups. Numbers on upper right corner denote Pearson's correlation coefficients. The histograms on the diagonal are frequency of % methylation per cytosine for each pool. MBC demonstrate a shift to the left compared to the DFS and Healthy, indicating genome-wide hypomethylation. FIG. 8B Principal Component Analysis (PCA) of the methylation profiles of each cfDNA pool, showing PC1 and PC2 for each sample. Samples closer to each other in clustering or principal component space are similar in their methylation profiles.

FIG. 9: WGBS identifies 21 gene DNA hypermethylation signature associated with MBC derived from largely European American women. Circos plot is graphing the target CpG Islands for each gene (left panel). Inner circle (red) is MBC, middle circle (green) is DFS and outer circle (blue) is Healthy subjects. Integrated genomic viewer of higher resolution snapshot of RUNX3 hotspot (right panel). Color codes same as circos.

FIGS. 10A-10B: bAmplicon-seq analysis in 30 individual samples for 8 hotspots regions. Percent methylation (FIG. 10A) and coverage for 3680 CpG loci (FIG. 10B) are plotted. Table summarizes % methylation statistics for 3680 CpG loci assayed across the dataset. 80% of loci in H samples had methylation values <5% demonstrating the potential for high signal to noise and sensitivity of the test.

FIG. 11: Bisulfite Primer PCR workflow.

FIG. 12: Example H&E images of two breast to brain metastases PDXs and associated metastases (*) (CM01, CM16) or (HCI011). All PDXs were grown in the lab. Note that in CM01 and CM16 were derived from brain metastasis patients but displayed additional sites of metastases in mice. Sites of involvement in mice mirrored the patient's sites of metastasis.

FIGS. 13A-13B: MSP results showing RUNX3 hotspot methylation in 18 PDXs. FIG. 13A Methylated (M) and unmethylated (U) primers indicate methylation + and − tumors. FIG. 13B Methylation primers used to show correlation of mouse tissue DNA with matching cfDNA extracted from plasma in one RUNX3 + and − models.

FIG. 14. Schema for patient accrual and treatment and time timing for blood collection that will and analyzed by CpG4C test.

FIG. 15. Possible Outcomes for CpG4C positive or negative blood tests in breast cancer patients after neoadjuvant therapy in the pre-metastatic setting.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cell” includes a plurality of cells.

### Definitions

The following definitions assist in defining the meets and bounds of the inventions as described herein.

The term “about” when used before a numerical designation, e.g., temperature, time, amount, concentration, and such other, including a range, indicates approximations which may vary by (+) or (−) 10%, 5% or 1%.

The terms “administering” or “administration” in reference to delivering engineered vesicles to a subject include any route of introducing or delivering to a subject the engineered vesicles to perform the intended function. Administration can be carried out by any suitable route, including orally, intranasally, parenterally (intravenously, intramuscularly, intraperitoneally, or subcutaneously), intracranially, or topically. Additional routes of administration include intraorbital, infusion, intraarterial, intracapsular, intracardiac, intradermal, intrapulmonary, intraspinal, intrasternai, intrathecal, intrauterine, intravenous, subarachnoid, subcapstilar, subcutaneous, transtnucosal, or transtracheal. Administration includes self-administration and the administration by another.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

In the context of a nucleic acid such as DNA, “cell-free” refers to a fragment of DNA or other nucleic acid that is freely circulating (i.e. not associated with a cell) in the blood stream, lymphatic system, or in the peritoneal fluid. Circulating tumor DNA is a form of cell-free DNA that is of tumor origin and/or originated from circulating tumor cells. Circulating tumor DNA may be shed from primary tumors, actively released from tumor cells, or result from apoptosis or necrosis of tumor cells. In some embodiments, the average size of a cell-free DNA fragment may correspond to the number of base pairs that wrap around a nucleosome (about 130 base pairs to about 170 base pairs, with or without a linker). In the context of a sample, “cell-free” refers to an isolated sample substantially free of cells. Cells may be actively removed from the sample by any method known in the art including, but not limited to centrifugation, column separation, and filtration. In some aspects, the sample may be of a type that does not contain many cells (e.g. plasma, saliva, urine, peritoneal fluid).

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.goviblast/Blast.cgi. Biologically equivalent polynucleotides are those having the specified percent homology and encoding a polypeptide having the same or similar biological activity.

As used herein, “CpG” refers generally to a dinucleotide consisting of a cytosine (C) nucleotide bound to a guanine (G) nucleotide through a phosphate (p) bond in a linear sequence of bases in the 5′ to 3′ direction. The cytosine residue of a CpG in a DNA sequence can be methylated at position C5 to form 5′methylcytosine. Methylation of CpGs in a DNA sequence can result in changes in access to the methylated DNA and regulatory effects including but not limited to repression of gene transcription, repression of transposable elements, genomic imprinting, and X-chromosome inactivation. Aberrant DNA methylation has also been associated with a variety of diseases such as cancer, imprinting disorders (e.g. Prader-Willi syndrome), Fragile X syndrome, and systemic lupus erythematosus. Global hypomethylation in cancer cells can contribute to genomic instability. Gene-specific hypermethylation at CpG islands near promoters can result in silenced transcription in cancer cells.

The term “suspected of having or developing cancer” intends a subject with one or more signs or symptoms of cancer or a history of having cancer. Signs and symptoms of cancer include but are not limited to skin changes, such as: a new mole or a change in an existing mole, a sore that does not heal; breast changes, such as: change in size or shape of the breast or nipple, change in texture of breast skin, a thickening or lump on or under the skin; hoarseness or cough that does not go away; changes in bowel habits; difficult or painful urination; problems with eating, such as: discomfort after eating, a hard time swallowing, changes in appetite; weight gain or loss with no known reason; abdominal pain; unexplained night sweats; unusual bleeding or discharge, including: blood in the urine, vaginal bleeding, blood in the stool; and feeling weak or very tired. Symptoms of breast cancer include but are not limited to the presence of a lump in the breast, bloody discharge from the nipple, discomfort, inverted nipple, redness, swollen lymph nodes and changes in the shape or texture of the nipple or breast.

As used herein, the term “early stage cancer” intends a cancer or tumor that is early in its growth, and may not have spread to other parts of the body. In some embodiments, an early stage cancer is a stage 0, stage I, or stage II cancer.

There are 3 known types of stage 0 breast cancers: ductal carcinoma in situ (DCIS), lobular carcinoma in situ (LCIS), and Paget disease of the nipple. DCIS is a noninvasive condition in which abnormal cells are found in the lining of a breast duct. The abnormal cells have not spread outside the duct to other tissues in the breast. LCIS is a condition in which abnormal cells are found in the lobules of the breast. Paget disease of the nipple is a condition in which abnormal cells are found in the nipple only. In breast cancer, Stage I is divided into stages IA and IB. In stage IA, the tumor is 2 centimeters or smaller. Cancer has not spread outside the breast. In stage IB, small clusters of breast cancer cells (larger than 0.2 millimeter but not larger than 2 millimeters) are found in the lymph nodes and either: (1) no tumor is found in the breast; or (2) the tumor is 2 centimeters or smaller. Stage II is also divided into stages: IIA and IIB. In stage IIA, (1) no tumor is found in the breast or the tumor is 2 centimeters or smaller. Cancer (larger than 2 millimeters) is found in 1 to 3 axillary lymph nodes or in the lymph nodes near the breastbone (found during a sentinel lymph node biopsy); or (2) the tumor is larger than 2 centimeters but not larger than 5 centimeters. Cancer has not spread to the lymph nodes. In stage IIB, the tumor is (1) larger than 2 centimeters but not larger than 5 centimeters. Small clusters of breast cancer cells (larger than 0.2 millimeter but not larger than 2 millimeters) are found in the lymph nodes; or (2) larger than 2 centimeters but not larger than 5 centimeters. Cancer has spread to 1 to 3 axillary lymph nodes or to the lymph nodes near the breastbone (found during a sentinel lymph node biopsy); or (3) larger than 5 centimeters. Cancer has not spread to the lymph nodes.

As used herein, the term “genomic region” refers to a specific locus in a subject's genome. In some embodiments, the size of the genomic region can range from one base pair to 10⁷ base pairs in length. In particular embodiments, the size of the genomic region is between 10 base pairs and 10,000 base pairs.

As used herein, the term “normal reference standard” intends a control level, degree, or range of DNA methylation at a particular genomic region or gene in a sample that is not associated with cancer. The term “normal reference cutoff value” refers to a control threshold level of DNA methylation at a particular genomic region or gene or a differential methylation value (DMV). In some embodiments, DNA methylation levels enriched above the normal reference cutoff value are associated with having or developing cancer. In some embodiments, DNA methylation levels at or below the normal reference cutoff value are associated with not having or developing cancer.

As used herein, the term “cancer recurrence” intends a cancer that has returned after a period of time during which the cancer could not be detected. The cancer may come back to the same place as the original (primary) tumor or to another place in the body.

“CpG island” refers to a region of DNA with a high frequency and/or enrichment of CpG sites. Algorithms can be used to identify CpG islands (Han, L. et al. (2008) Genome Biology, 9(5): R79). Generally, enrichment is defined as a ratio of observed-to-expected CpGs for a given DNA sequence greater than about 40%, about 50%, about 60%, about 70%, about 80%, or about 90-100%. In some embodiments, CpGs listed herein are numbered as reported in the hg19 genome build (as viewed in the Integrated Genomic Viewer (James T. Robinson et al. Integrative Genomics Viewer. Nature Biotechnology 29, 24-26 (2011)), last accessed Aug. 17, 2017). As used herein, a “region” refers to a CpG enriched genomic region comprising at least 10 CpGs.

As used herein, the term “DNA methylation” intends the presence of one or more methyl groups on a DNA molecule. In some embodiments, the DNA molecule is methylated at the 5-carbon of the cytosine ring resulting in 5-methylcytosine (5-mC). In some embodiments, 5-mC occurs in the context of paired symmetrical methylation of a CpG site, in which a cytosine nucleotide is located next to a guanidine nucleotide. In the context of DNA methylation, the term “level” refers to the amount or frequency of methylated DNA residues present or detected in a particular genomic region or gene.

A “gene” refers to a polynucleotide containing at least one open reading frame (ORF) that can be transcribed into an RNA (e.g. miRNA, siRNA, mRNA, tRNA, and rRNA) that may encode a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

A “gene product” or alternatively a “gene expression product” refers to the RNA when a gene is transcribed or amino acid (e.g., peptide or polypeptide) generated when a gene is transcribed and translated.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced there from.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The terms “patient,” “subject,” or “mammalian subject” are used interchangeably herein and include any mammal in need of the treatment or prophylactic methods described herein (e.g., methods for the treatment or prophylaxis of cancer, hemophilia). Such mammals include, particularly humans (e.g., fetal humans, human infants, human teens, human adults, etc.). Other mammals in need of such treatment or prophylaxis can include non-human mammals such as dogs, cats, or other domesticated animals, horses, livestock, laboratory animals (e.g., lagomorphs, non-human primates, etc.), and the like. The subject may be male or female.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, preferably, a human. In some embodiments, the sample is obtained in a non-invasive or minimally invasive manner.

The terms “treatment,” “treat,” “treating,” etc. as used herein, include but are not limited to, alleviating a symptom of a disease or condition (e.g., cancer) or a condition associated with cancer and/or reducing, suppressing, inhibiting, lessening, ameliorating or affecting the progression, severity, and/or scope of the disease or condition. “Treatments” refer to one or both of therapeutic treatment and can separately relate to prophylactic or preventative measures as desired. Prevention may not be obtainable for certain diseased or conditions and for those conditions, prevention is excluded from the term treatment. Subjects in need of treatment include those already affected by a disease or disorder or undesired physiological condition as well as those in which the disease or disorder or undesired physiological condition is to be prevented.

“Detecting” as used herein refers to determining the presence and/or degree of methylation in a nucleic acid of interest in a sample. Detection does not require the method to provide 100% sensitivity and/or 100% specificity.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “identify” or “identifying” is to associate or affiliate a patient closely to a group or population of patients who likely experience the same or a similar clinical outcome, course of disease, life expectancy, clinical response, clinical parameter, disease progression, disease recurrence, metastasis, or clinical response to a therapy. In some aspects, “identifying” refers to discovery and/or selection of a screening marker, diagnostic marker, predictive marker, prognostic markers, or panel of markers (e.g. a marker “signature”) specific for a disease or condition.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov. Typically, a patient is given a subsequent chemotherapy regimen because the patient did not show a positive clinical or sub-clinical response to the first line therapy or the first line therapy has stopped.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), objective response rate (RR or ORR), toxicity or side effect.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the term “cancer” intends a malignant phenotype characterized by the uncontrolled proliferation of malignant cells. A “tumor” intends a neoplasm that may be benign or malignant. As used herein, “cancer cells” and “tumor cells” are used interchangeably to refer to malignant neoplasmic cells. The methods and compositions of this disclosure are useful for the treatment, diagnosis, and screening of cancers including but not limited to lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma, or recurrence or metastasis of lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma. The cancer can be metastatic, non-metastatic and pre-clinical.

The term “chemotherapy” encompasses cancer therapies that employ chemical or biological agents or other therapies, such as radiation therapies, e.g., a small molecule drug or a large molecule, such as antibodies, RNAi and gene therapies.

### Methods

The methods described herein are useful in the assistance of an animal, a mammal or yet further a human patient. For the purpose of illustration only, a mammal includes but is not limited to a human, a simian, a murine, a bovine, an equine, a porcine or an ovine subject. In some embodiments, the subject is a patient suspected of having a disease or condition.

### Identification of Novel Biomarkers for Disease

Described herein is a method for identifying screening, predictive, prognostic, or diagnostic markers for a disease, the method comprising, consisting of, or consisting essentially of: a) determining the methylation profile of a pool of cell free DNA samples isolated from subjects with the disease; b) determining the methylation profile of a pool of cell free DNA samples isolated from disease-free subjects or a normal reference standard; wherein each pool consists of equal amounts of cell free DNA; c) comparing the methylation profiles determined in steps a) and b); and d) selecting differentially methylated regions with greater than 40% differential value. In some embodiments, the samples are isolated from solid tumors and corresponding disease-free tissue, or a disease free subject.

Sample pool preparation: First, nucleic acids are extracted from a sample isolated from the subject. In some embodiments, the sample is cell-free. In some embodiments, the nucleic acids isolated from the sample are cell-free (e.g. cell-free DNA or cell-free RNA). In some aspects, the sample isolated from the subject is a non-invasive or minimally invasive sample. Non-limiting examples of non-invasive or minimally invasive samples include whole blood, plasma, serum, urine, feces, saliva, buccal mucosa, sweat, and tears.

Any method known in the art can be used to extract the nucleic acids from the sample isolated from the subject. (e.g. with MagMAX™ Cell-free DNA Isolation Kit (Thermofisher)). In some embodiments, more than one sample can be isolated from the subject and pooled to create a single test sample. In some embodiments, pooling may be performed before or after the nucleic acid extraction.

Preparation of control samples: A normal reference standard or reference cutoff value is used for comparative methylation studies. In some embodiments, a normal reference standard is prepared from one or more samples isolated from one or more subjects that have not been diagnosed with cancer and are not suspected of having cancer. In other embodiments, a normal reference standard is prepared from one or more samples isolated from a corresponding disease-free tissue (i.e. normal tissue) of a subject suspected of having or developing cancer. In some embodiments, a reference cutoff value of DNA methylation is determined by detecting the level of DNA methylation in one or more reference samples.

In some embodiments, the number of samples per sample pool is from 2 to 5, 2 to 10, 2 to 15, 2 to 20, 2 to 30, 2 to 40, 2 to 50, 2 to 75, 2 to 100, 2 to 150, 2 to 200, 2 to 300, 2 to 400, 2 to 500, 2 to 1000, 2 to 1000, 5 to 10, 5 to 15, 5 to 20, 5 to 50, 10 to 20, 10 to 30, 10 to 40, 10 to 50, 10 to 75, 10 to 100, 10 to 150, 10 to 200, 10 to 300, 10 to 400, 10 to 500, 100 to 200, 100 to 300, 100 to 400, 100 to 500, 100 to 1000, 500 to 1500, 1000 to 2000, 1000 to 3000, 1000 to 4000, 1000 to 5000, 1000 to 6000, 1000 to 7000, 1000 to 8000, 1000 to 9000, 1000 to 10000, or 5000 to 10000. In some embodiments, samples from a large number of subjects enrolled in a multi-institution clinical study are pooled. For example, samples may be pooled from a cohort of one million patients. The amount of nucleic acid in each pool should be normalized so that each pool contains an equivalent or nearly equivalent amount of nucleic acid prior to performing methylation analysis.

Determination of methylation level or methylation profile in sample pools. Differential methylation analysis in combination with DNA sequencing is performed to determine the methylation profile of the sample pools. A methylation profile includes all data generated by a methylation assay including but not limited to nucleotide sequence data, identification of methylated cytosine residues in the nucleotide sequences, frequency of methylation, degree of methylation, relative ratios of DNA fragments, relative enrichment of methylation, density of methylation, integrity of DNA fragments, and other data and outputs known in the art. Data may be further processed by algorithms and/or software to determine the differential values (i.e. differential methylation value) and identify differentially methylated regions (DMRs). Differential methylation value may be calculated by methods known in the art (see, e.g. Hovestadt, V., et al. (2014). Decoding the regulatory landscape of medulloblastoma using DNA methylation sequencing. Nature, 510(7506), 537-541). In some aspects, Metilene, a software program for calling differentially methylated regions may be used (Juhling et al. (2015) Genome Research doi: 10.1101/gr.196394.115). Metilene utilizes an algorithm to identify differentially methylated regions within whole genome and targeted sequencing data.

In one aspect, methylation analysis is performed using whole genome bisulfite sequencing (WGBS). It is important that equal or nearly equivalent amounts of cell free DNA from each pooled sample is used for WGBS. Commercial library prep kits may be used to prepare the pools for WGBS (e.g. Nugen or MethylKit). Sequencing is performed using a sequencing platform (e.g. HiSeq, Illumina, CA, USA). Differential methylation region analysis (i.e. identify regions of at least 10 CpG sites) and select all regions with greater than 40% or greater than 50% differential value. The reference pool or the pool of samples isolated from normal subjects or corresponding normal tissues should have absolute methylation levels of less than about 10%.

In one aspect, the method further comprises validation of the selected regions. Validation may be performed using one or more of targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

In some aspects, validation comprises targeted amplicon bisulfite sequencing.

The following exemplary steps are performed to validate selected markers with targeted bisulfite Amplicon sequencing (bAmplicon-seq).

1) Primers are designed to bisulfite converted DNA using BiSearch or bisulfite primer seeker. Allow 1-3 degenerate bases in first third of primer. Primers are typically 25-30 nucleotides long and amplicons range from 60-500 base pairs or 100-250 base pairs. Amplicons are optimally below 180 base pairs. 2-3 primer pairs are designed per region. Sets of primer pairs are designed to amplify both forward and reverse strands of DNA, when possible.

2) Test primers and optimize primer melting temperature (Tm).

3) Assess primer bias for unmethylated DNA by mixing commercially bought 100% and 0% methylated DNA. Test ratio of 100%, 50% and 0% methylation. Assess melting curves and look for methylated and unmethylated peaks and their ratio shift.

4) Pick the best primers and redesign if needed.

5) Optimize primer multiplex PCR conditions. Multiplex 5-10 primers per plex.

6) After multiplex PCR, perform singleton PCR analysis to ensure co-efficiency of each primer in plex. Primers should amplify each amplicon at near equal levels. This is asses using PCR ct values. Adjust primer concentrations to correct any primer off-sets.

7) It was determined empirically that 3ng input cell-free DNA is optimal for 6plex PCR.

8) After multiplex PCR, clean-up product with PCR NucleoSpin column (Macherey-Nagel). Clean high and low molecular weight artefacts with 2-sided SPRI beads.

9) Quantitate multiplex PCR product.

10) Use 40 ng of multiplex PCR product into library preparation step.

11) Use KAPA Hyper Prep Kit (Kapa Bioscience) for library preparation. Perform 5 rounds of post-library PCR. Use SureSelect XT2 pre-capture adapters.

12) Clean-up and quantitate library preps.

13) Pool libraries using equimolar of each sample to make a final pooled sample at 4 nM concentration.

14) Sequence libraries on Illumina Miseq (2×150 base pair design). Use 15% PhiX spike-in. Sequence depth should exceed 5000× per amplicon.

15) Perform data analysis.

Alignment of bisulfite converted DNA is performed using a software program such as Bismark (Krueger, F. et al. (2011) Bioinformatics, 27(11): 157171). Bismark performs both read mapping and methylation calling in a single step and its output discriminates between cytosines in CpG, CHG and CHH contexts. Bismark is released under the GNU GPLv3+ licence. The source code is freely available at bioinformatics.bbsrc.ac.uk/projects/bismark/ (last accessed Aug. 17, 2017).

In some aspects, the disease is one of lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma, or recurrence or metastasis of lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma.

### Diagnostic Methods

Provided herein is a method for determining whether a subject is likely to have or develop cancer or cancer recurrence, the method comprising, consisting of, or consisting essentially of: (a) determining the level of DNA methylation at a genomic region within 10³ kb of one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty genes selected from the genes listed in Table 1 in a sample isolated from the subject; (b) comparing the level of DNA methylation in the sample to the level of DNA methylation in a sample isolated from a cancer-free subject, a normal reference standard, or a normal reference cutoff value; and (c) determining that the subject is likely to have or develop cancer or cancer recurrence if the level of DNA methylation in the sample derived from the subject is greater than the level of DNA methylation in the sample isolated from a cancer-free subject, a normal reference standard, or a normal reference cutoff value. In some aspects, greater than thirty genes are selected. In some aspects, the DNA is cell-free DNA and/or the sample is a cell-free sample.

TABLE 1

| | | | Chromosomal | |
|---|---|---|---|---|
| | Entrez | Chr. | Location of Gene | |
| Gene | Gene ID | # | (GRCh38) | Gene Name |
| RRAGC | 64121 | 1 | 38,838,198-38,859,823 | Ras-related GTP binding C |
| RNF207 | 388591 | 1 | 6,206,176-6,221,299 | ring finger protein 207 |
| CAMTA1 | 23261 | 1 | 6,785,324-7,769,706 | calmodulin binding transcription activator 1 |
| GP5 | 2814 | 3 | 194,394,821-194,398,354 | glycoprotein V platelet |
| IL17RE | 132014 | 4 | 9,902,612-9,916,402 | interleukin 17 receptor E |
| BANK1 | 55024 | 4 | 101,411,286-102,074,812 | B-cell scaffold protein with ankyrin repeats 1 |
| LIMCH1 | 22998 | 4 | 41,359,607-41,700,044 | LIM and calponin homology domains 1 |
| COX7B2 | 170712 | 4 | 46,734,827-46,909,235 | cytochrome c oxidase subunit 7B2 |
| ANKRD33B | 651746 | 5 | 10,564,330-10,657,816 | ankyrin repeat domain 33B |
| LOC648987 | 648987 | 5 | 43,014,729-43,067,439 | Uncharacterized human LOC648987 |
| ATG9B | 285973 | 7 | 151,012,209-151,024,499 | autophagy related 9B |
| NR_027387 | 100128822 | 7 | 152,464,124-152,465,545 | Homo sapiens long intergenic non-protein |
| | | | | coding RNA 1003 (LINC01003) |
| HOXA2 | 3199 | 7 | 27,100,354-27,102,811 | Homeobox A2 |
| LOC401321 | 401321 | 7 | 32,758,286-32,762,924 | Homo sapiens long intergenic non-protein |
| | | | | coding RNA 997 (LINC00997) |
| KBTBD2 | 25948 | 7 | 32,868,172-32,894,131 | kelch repeat and BTB domain containing 2 |
| SPAG1 | 6674 | 8 | 100,157,906-100,259,278 | sperm associated antigen 1 |
| MAFA | 389692 | 8 | 143,419,182-143,430,406 | MAF bZIP transcription factor A |
| ANK1 | 286 | 8 | 41,653,220-41,896,762 | ankyrin 1 |
| PBX3 | 5090 | 9 | 125,747,345-125,967,377 | Pbx homeobox 3 |
| FUBP3 | 8939 | 9 | 130,578,965-130,638,352 | far upstream element binding protein 3 |
| RABL6 | 55684 | 9 | 136,807,943-136,841,187 | RAB, member RAS oncogene family like 6 |
| C9ORF139 | 401563 | 9 | 137,027,464-137,037,957 | chromosome 9 open reading frame 139 |
| DIP2C | 22982 | 10 | 274,190-689,668 | disco interacting protein 2 homolog C |
| CHFR | 55743 | 12 | 132,822,187-132,956,304 | checkpoint with forkhead and ring finger domains |
| ZNF605 | 100289635 | 12 | 132,918,308-132,956,306 | zinc finger protein 605 |
| DZIP1 | 22873 | 13 | 95,578,202-95,644,703 | DAZ interacting zinc finger protein 1 |
| SLC35F4 | 341880 | 14 | 57,563,922-57,982,194 | solute carrier family 35 member F4 |
| ARHGAP23 | 57636 | 17 | 38,419,280-38,512,392 | Rho GTPase activating protein 23 |
| STAC2 | 342667 | 17 | 39,210,536-39,225,872 | SH3 and cysteine rich domain 2 |
| STAC2 | 342667 | 17 | 39,210,536-39,225,872 | SH3 and cysteine rich domain 2 |
| ACSF2 | 80221 | 17 | 50,426,158-50,474,845 | acyl-CoA synthetase family member 2 |
| UNC13A | 23025 | 19 | 17,601,328-17,688,365 | unc-13 homolog A |
| PBX4 | 80714 | 19 | 19,561,707-19,618,916 | pbx homeobox 4 |
| FUZ | 80199 | 20 | 49,806,869-49,817,376 | fuzzy planar cell polarity protein |
| ISM1 | 140862 | 20 | 13,221,771-13,300,651 | isthmin 1 |
| BMP2 | 650 | 3 | 6,767,664-6,780,280 | bone morphogenetic protein 2 |
| LOC286647 | | X | | Uncharacterized human LOC286647 |

Also provided is a method for detecting the level of DNA methylation in a sample isolated from a subject suspected of having or developing cancer or early stage cancer, the method comprising, consisting of, or consisting essentially of determining the level of DNA methylation at a genomic region within 10³ kb of one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty genes selected from the genes listed in Table 1 in the sample. In some aspects, the method further comprises comparing the measured level of DNA methylation in the sample to the level of DNA methylation in a sample isolated from a cancer free subject, a normal reference standard, or a normal reference cutoff value. In some aspects, greater than thirty genes are selected. In some aspects, the DNA is cell-free DNA and/or the sample is a cell-free sample.

In one aspect, the level of DNA methylation is determined at one or more CpG islands and/or regions within 10³ kb of the 5′ or 3′ end of the selected gene or genes in Table 1. In other aspects, the level of DNA methylation is determined at a region within 900 kb, 800 kb, 700 kb, 600 kb, 500 kb, 400 kb, 300 kb, 200 kb, 100 kb, 50 kb, 10 kb, or 5 kb of the 5′ or 3′ end (i.e. upstream or downstream) of the selected gene or genes.

In some aspects, the level of DNA methylation is determined at a region within the selected gene or genes. Nonlimiting examples include a region within an untranslated region (UTR) of the selected gene or genes, a region within 1.5 kb upstream of the transcription start site of the selected gene or genes, and a region within the first exon of the selected gene or genes.

Also provided herein is a method for determining whether a subject is likely to have or develop cancer or early stage cancer, the method comprising, consisting of, or consisting essentially of: (a) determining the level of DNA methylation at one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty regions selected from the regions listed in Table 2 in a sample isolated from the subject; (b) comparing the level of DNA methylation in the sample to the level of DNA methylation in a sample isolated from a cancer-free subject, a normal reference standard, or a normal reference cutoff value; and (c) determining that the subject is likely to have or develop cancer or cancer recurrence if the level of DNA methylation in the sample derived from the subject is greater than the level of DNA methylation in the sample isolated from a cancer-free subject, a normal reference standard, or a normal reference cutoff value. In some aspects, greater than thirty regions are selected. In some aspects, the DNA is cell-free DNA and/or the sample is a cell-free sample.

TABLE 2

| Methylation Region | | Closest |
|---|---|---|
| (hg19 genome build) | CpG | Gene |
| chr1: 39,044,074-39,044,225 | CPG59 | RRAGC |
| chr1: 6,268,888-6,269,045 | CPG88 | RNF207 |
| chr1: 7,765,055-7,765,179 | CPG157 | CAMTA1 |
| chr3: 194,117,921-194,118,045 | CPG109 | GP5 |
| chr3: 9,957,033-9,957,468 | CPG95 | IL17RE |
| chr4: 102,712,059-102,712,200 | CPG35 | BANK1 |
| chr4: 41,646,367-41,646,493 | CPG32 | LIMCH1 |
| chr4: 46,726,427-46,726,601 | | COX7B2 |
| chr5: 10,565,517-10,565,682 | CPG253 | ANKRD33B |
| chr5: 43,018,031-43,018,972 | CPG67 | LOC648987 |
| chr7: 150,715,883-150,715,989 | CPG120 | ATG9B |
| chr7: 152,161,438-152,161,508 | CPG132 | NR_027387 |
| chr7: 27,141,743-27,141,932 | | HOXA2 |
| chr7: 32,801,782-32,802,525 | CPG40 | LOC401321 |
| chr7: 32,930,792-32,930,842 | CPG109 | KBTBD2 |
| chr8: 101,225,311-101,225,367 | CPG72 | SPAG1 |
| chr8: 144,511,850-144,512,138 | CPG294 | MAFA |
| chr8: 41,655,108-41,655,453 | CPG79 | ANK1 |
| chr9: 128,510,274-128,510,341 | CPG233 | PBX3 |
| chr9: 133,454,823-133,454,962 | CPG83 | FUBP3 |
| chr9:139,715,901-139,716,003 | CPG96 | RABL6 |
| chr9: 139,925,051-139,925,313 | COG58 | C9ORF139 |
| chr10: 735,378-735,552 | CPG100 | DIP2C |
| chr12: 133,481,446-133,481,616 | | CHFR |
| chr12: 133,481,446-133,481,616 | | ZNF605 |
| chr13: 96,293,984-96,294,377 | CPG89 | DZIP1 |
| chr14: 58,332,639-58,332,759 | CPG131 | SLC35F4 |
| chr17: 36,666,487-36,666,582 | COG144 | ARHGAP23 |
| chr17: 37,366,246-37,366,533 | CPG54 | STAC2 |
| chr17: 37,381,269-37,381,871 | CPG150 | STAC2 |
| chr17: 48,546,161-48,546,934 | CPG116 | ACSF2 |
| chr19: 17,716,756-17,717,092 | CPG93 | UNC13A |
| chr19: 19,729,144-19,729,553 | CPG60 | PBX4 |
| chr19: 50,312,537-50,312,694 | CPG66 | FUZ |
| chr20: 13,200,413-13,200,789 | CPG194 | ISM1 |
| chr20: 6,748,289-6,748,421 | CPG169 | BMP2 |
| chrX: 130,929,860-130,930,244 | CPG26 | LOC286647 |

*CpG islands as identified using Integrated Genomics Viewer (James T. Robinson et al. Integrative Genomics Viewer. Nature Biotechnology 29, 24-26 (2011)), last accessed Aug. 17, 2017.

In some aspects, the DNA methylation level is determined at one or more of the following genes or regions listed in Table 2 selected from ARHGAP23, ACSF2, RRAGC, RNF207, GPS, ANKRD33B, LOC648987, ATG9B, LOC401321, ANK1, PBX3, DIP2C, CHFR, ZNF605, STAC2, STAC2, ISM1, and LOC286647. In particular embodiments, the DNA methylation level is determined at ARHGAP23 and/or ACSF2, and optionally one or more genes or regions identified in Table 2 and/or Table 3.

In some aspects, the DNA methylation level is determined at one or more of the genes or regions listed in Table 3.

TABLE 3

| Methylation Region | | Closest |
|---|---|---|
| (hg19 genome build) | CpG | Gene |
| chr1: 119,522,297-119,522,685 | | TBX15 |
| chr1: 6,508,634-6,508,912 | | ESPN |
| chr1: 39,044,074-39,044,222 | CPG59 | |
| chr1: 39,269,706-39,269,850 | CPG72 | |
| chr1: 150,122,865-150,123,881 | | PLEKHO1 |
| chr1: 226,736,415-226,736,530 | CPG122 | C1ORF95 |
| chr1: 228,651,389-228,652,669 | CPG84 | |
| chr1: 42,383,685-42,383,856 | CPG38 | HIVEP3 |
| chr2: 220,313,284-220,313,454 | | SPEG8 |
| chr2: 220,313,294-220,313,436 | | SPEG8 |
| chr2: 208,989,125-208,989,413 | | NR_038437 |
| chr2: 162,100,925-162,101,769 | CPG24, | TANK |
| chr2: 131,792,795-131,792,937 | | ARHGEF4 |
| chr2: 468,028-468,289 | CPG79, | |
| chr3: 125,076,002-125,076,434 | | ZNF148 |
| chr3: 194,117,552-194,119,057 | | GP5 |
| chr3: 99,595,058-99,595,326 | | MIR548G |
| chr4: 46,726,419-46,726,619 | | COX7B2 |
| chr4: 8,895,441-8,895,846 | CPG254 | |
| chr4: 13,549,015-13,549,160 | | LOC285548 |
| chr4: 25,235,927-25,236,058 | | PI42KB |
| chr4: 30,723,649-30,723,941 | | PCDH7 |
| chr4: 46,726,525-46,726,603 | | COX7B2 |
| chr4: 153,858,813-153,858,916 | CPG213, | FHDC1 |
| chr5: 94,955,846-94,956,706 | | GPR150 |
| chr5: 1,445,269-1,445,490 | | SLC6A3 |
| chr6: 117,591,888-117,592,164 | | VGLL2 |
| chr6: 6,002,430-6,002,857 | | NRN1 |
| chr7: 155,167,043-155,167,243 | CPG277, | BLACE |
| chr7: 151,106,717-151,106,910 | | WDR86 |
| chr7: 27,204,874-27,205,029 | | HOXA9 |
| chr8: 55,379,115-55,379,416 | CPG131, | SOX17 |
| chr9: 133,308,833-133,309,057 | CPG99, | ASS1 |
| chr10: 45,914,402-45,914,709 | | ALOX5 |
| chr10: 77,156,043-77,156,222 | CPG987, | ZNF503 |
| chr11: 75,379,637-75,379,770 | | MAP6 |
| chr11: 725,576-725,843 | | EPS8L2 |
| chr12: 58,021,185-58,021,918 | | B4GALANT1 |
| chr13: 96,204,915-96,205,232 | | CLDN10 |
| chr13: 29,393,957-29,394,126 | CPG109 | |
| chr14: 38,724,432-38,725,600 | | CLEC14A |
| chr15: 66,914,674-66,914,722 | CGG65 | |
| chr15: 65,116,372-65,116,575 | | PIF1 |
| chr16: 87,636,189-87,636,318 | | JPH3 |
| chr16: 51,185,202-51,185,325 | | SALL1 |
| chr17: 1,960,496-1,960,610 | | HIC1 |
| chr17: 7,554,926-7,555,051 | | ATP1B2 |
| chr17: 36,714,476-36,714,611 | | SRCIN1 |
| chr17: 44,337,407-44,337,726 | CPG51 | |
| chr18: 70,522,481-70,548,676 | | NETO1 |
| chr19: 30,716,841-30,717,033 | CPG265 | |
| chr19: 50,030,948-50,031,354 | | RCN3 |
| chr22: 19,711,302-19,711,474 | | SEPT5-GP1BB |

In some aspects, the DNA methylation level is determined at one or more of the genes or regions listed in Tables 2 and/or 3.

In some aspects, the DNA methylation level is determined with targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

In another aspect, the method further comprises performing one or more of targeted bisulfite amplicon sequencing, bisulfite DNA treatment, whole genome bisulfite sequencing, bisulfate conversion combined with bisulfate restriction analysis (COBRA), bisulfate PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

In some aspects, the sample isolated from the subject is a non-invasive or minimally invasive sample. Non-limiting examples include whole blood, plasma, serum, urine, feces, saliva, buccal mucosa, sweat, or tears. In a further aspect, the sample is cell-free and/or comprises cell-free DNA.

In some aspects, the methods determine whether a subject is likely to have or develop lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma, or recurrence or metastasis of lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, Kaposi sarcoma. In particular embodiments, the methods determine whether a subject is likely to have or develop breast cancer.

### Targeted Bisulfite Amplicon Sequencing

Targeted bisulfite amplicon sequencing is performed, for example, on Illumina's MiSeq platform. This nascent, deep-sequencing strategy allows for sensitive detection of DNA methylation in low-input samples such as plasma. Exemplary methods for performing this assay are described in Masser et al. (2015) J Vis Exp. (96): 52488, incorporated herein by reference.

Briefly, nucleic acids are isolated from the sample and quantified. Bisulfite conversion of DNA (e.g. cell-free DNA) is performed using, for example, a commercially available kit such as EZ DNA Methylation™ Kit (available from Zymo Research, Tustin, Calif., USA), EpiMark® Bisulfite Conversion Kit (available from New England Biolabs, Inc., Ipswich, Mass., USA), and Epitect Bisulfite Kits (available from Qiagen, Germantown, Md., USA). Bisulfite conversion changes the unmethylated cytosines into uracils. These uracils are subsequently converted to thymines during later PCR amplification.

Bisulfite converted DNA is amplified by bisulfite specific PCR using a polymerase capable of amplifying bisulfite converted DNA. DNA approximately 60-500 bp in length corresponding to the regions listed in Tables 1, 2, or 3 are amplified. Amplicons are visualized by PAGE electrophoresis. Alternatively, capillary electrophoresis with a DNA chip is used according to manufacturer's protocol.

Exemplary PCR primers for amplifying regions within 10³ kb of Bank1, LIMCH1, ANK1, and FUZ are provided below:

BANK1_1b+(F1):
(SEQ ID NO: 1)
TTAGTAGYGTTAGGTAAGGGGTTTGGGAG
BANK1_1b+(R):
(SEQ ID NO: 2)
CTCAAAAACRCCCTAACCTCAATACCC
BANK1_1b+(F2):
(SEQ ID NO: 3)
GGTTTAGYGTTTTTAGGTGGGTAG
BANK1_1b-(F1):
(SEQ ID NO: 4)
YGGTAGGATAAAAAGGAGAAGTTTTG
BANK1_1b-(R2):
(SEQ ID NO: 5)
ACCCAACRCCCCCAAATAAATAATC
LIMCH1_1c+(F):
(SEQ ID NO: 6)
GTAGTTYGGGAAGGGGGTAGTTTTTTAAG
LIMCH1_1c+(R):
(SEQ ID NO:7)
CCTCCTCACACCRCATATCAAACATACTAATACTCC
LIMCH1_1b-(F):
(SEQ ID NO: 8)
GTGATTGGYGGTGTGTTTTGGTTTTGGG
LIMCH1_1b-(R):
(SEQ ID NO: 9)
TAACCCRATTCAATAACATCACTAAAAAC
ANK1_1b+(F1):
(SEQ ID NO: 10)
AGAGTAGTYGGGGAGAGTTGAGTTTAGAGTTTAGAG
ANK1_1b+(R):
(SEQ ID NO: 11)
AAAATTCCCRCTTAATATTACTTCCCCTACACCCAAC
ANK1_1b+(F2):
(SEQ ID NO: 12)
ATTAGGTTATAYGTTGAGAGGGTAGTAAATGAAAGGG
ANK1_1b-(F1):
(SEQ ID NO: 13)
AGYGATTTTTAGATAAGTAGAAGAGGAGATG
ANK1_1b-(R):
(SEQ ID NO: 14)
CCTAAAAACCRCAAATTACAAAAACACCTCCTCC
ANK1_1b-(F2):
(SEQ ID NO: 15)
ATTTTTTTAGYGTGTGGTTTGATGTTTAATTTTGGG
FUZ_1+(F):
(SEQ ID NO: 16)
GGATTTGAAGTAGGGTATAGGTTGGGG
FUZ_1+(R):
(SEQ ID NO: 17)
RTACTACTCCCCTAACTAATAAAATCCCTACC
FUZ_1-(F):
(SEQ ID NO: 18)
YGTGTTGTTTTTTTGGTTGGTGGGGTTTTTG
FUZ_1-(R):
(SEQ ID NO: 19)
AAACCTAAAACAAAACACAAACTAAAACTCATC
FUZ_1b+(F1):
(SEQ ID NO: 20)
TTTTAGGTTYGGTAGTAGAGTTAGGGTTAGGAG
FUZ_1b+(R1):
(SEQ ID NO: 21)
CCRTACTACTCCCCTAACTAATAAAATCCCTAC
FUZ_1b+(F2):
(SEQ ID NO: 22)
GGGTTAGGAGTYGGTGTGGGATTTGAAGTAGGGTATAG
FUZ_1b+(R2):
(SEQ ID NO: 23)
AAACCRTACTACTCCCCTAACTAATAAAATCCC
FUZ_1b-(F1):
(SEQ ID NO: 24)
GTGGTAGTAATAGAGGGTTGGTGG
FUZ_1b-(R1):
(SEQ ID NO: 25)
ACCTAAAACAAAACACAAACTAAAACTCATC
FUZ_1b-(F2):
(SEQ ID NO: 26)
TYGTGTTGTTTTTTTGGTTGGTGGGGTTTTTG
FUZ_1b-(R2):
(SEQ ID NO: 27)
CTCCAAACTCRACAACAAAATCAAAATCAAAAACC
STAC2_1b+(F):
(SEQ ID NO: 28)
TYGGAGGGTATTTTTGGGTGGGTAAG
STAC2_1b+(R):
(SEQ ID NO: 29)
ACAAACRACAACATAACAAAAATCCCAAACCTCATCCC
STAC2_1b-(F1):
(SEQ ID NO: 30)
TTYGAGGAGGGTGGGGTTTGGGGAGAGTTAAAAGGG
STAC2_1b-(R1):
(SEQ ID NO: 31)
ACTAACCTCRAATAAATACTAAACCCTCCCAAACCC
STAC2_1b-(F2):
(SEQ ID NO: 32)
TTGGGGAGAGTTAAAAGGGGATTTGAGGAAAGTGG
STAC2_1b-(R2):
(SEQ ID NO: 33)
AAACTAACCTCRAATAAATACTAAACCCTCCCAAAC
STAC2_1b-(F3):
(SEQ ID NO: 34)
TAGGYGGTAATATGGTAGGGGTTTTAGG
STAC2_1b-(R3):
(SEQ ID NO: 35)
CTCRAAAAACACCTCTAAATAAACAAAATC
STAC2_2b+(F):
(SEQ ID NO: 36)
TATGGTTYGGGGAGAGGGGAGGAGAG
STAC2_2b+(R):
(SEQ ID NO: 37)
TACCRAAAACTAACTAAAAACAACCTCTAAAAAAC

A next generation sequencing library is prepared with the amplicons. Nonlimiting examples of methods for preparing the library include using a transposome-mediated protocol with dual indexing, and/or a kit (e.g. TruSeq Methyl Capture EPIC Library Prep Kit, Illumina, CA, USA, Kapa Hyper Prep Kit (Kapa Biosystems). Adapters such as TruSeq DNA LT adapters (Illumina) can be used for indexing. Sequencing is performed on the library using a sequencer platform (e.g. MiSeq or HiSeq, Illumina).

Bisulfite-modified DNA reads are aligned to a reference genome using alignment software (e.g., Bismark tool version 0.12.7). Differential methylation is calculated for specific loci/regions. In some embodiments, a differential methylation value (DMV) of about 10, about 15, about 18, about 20, about 22, about 25, about 30, about 35, about 40, about 45, about 50, about 55, or about 60 (in percent scale) is considered a differentially methylated locus (DML) or differentially methylated region (DMR). In some embodiments, a DMV of about 20 percent is considered a DML or DMR. In some embodiments, a P value less than about 0.05 is considered a DML or DMR.

The subject is determined to be likely to have or develop cancer or cancer recurrence if DNA methylation is enriched at the selected genes or regions as compared to the normal control sample, the reference standard, or the cutoff value. In some embodiments, the reference cutoff value is a DMV of about 10, about 15, about 18, about 20, about 22, about 25, about 30, about 35, about 40, about 45, about 50, about 55, or about 60 (in percent scale). In some embodiments, the reference cutoff value is about 40 percent.

In some embodiments, genes or regions located on the X and/or Y sex chromosomes are removed from the analysis.

### Therapy

The information obtained using the diagnostic methods described herein is useful for determining if a subject is likely to have or develop cancer or cancer recurrence. Based on the prognostic or diagnostic, or predictive information, a doctor can recommend a therapeutic protocol, useful for preventing or reducing the malignant mass, tumor, or metastasis in the subject or treating cancer in the subject. Thus, in some aspects, provided herein are methods of selectively treating a subject, the method comprising administering a therapy or treatment to a subject having previously determined to be likely to have or develop cancer or cancer recurrence. In some aspects, the subject was previously determined to have a particular methylation profile.

A patient's likely clinical outcome following a clinical procedure such as a therapy or surgery can be expressed in relative terms. For example, a patient having a particular methylation profile can experience relatively longer overall survival than a patient or patients not having the methylation profile. The patient having the particular methylation profile, alternatively, can be considered as likely to survive. Similarly, a patient having a particular methylation profile can experience relatively longer progression free survival, or time to tumor progression, than a patient or patients not having the methylation profile. The patient having the particular methylation profile, alternatively, can be considered as not likely to suffer tumor progression. Further, a patient having a particular methylation profile can experience relatively shorter time to tumor recurrence than a patient or patients not having the methylation profile. The patient having the particular methylation profile level, alternatively, can be considered as not likely to suffer tumor recurrence. Yet in another example, a patient having a particular methylation profile can experience relatively more complete response or partial response than a patient or patients not having the methylation profile. The patient having the particular methylation profile, alternatively, can be considered as likely to respond. Accordingly, a patient that is likely to survive, or not likely to suffer tumor progression, or not likely to suffer tumor recurrence, or likely to respond following a clinical procedure is considered suitable for the clinical procedure.

It is to be understood that information obtained using the diagnostic methods described herein can be used alone or in combination with other information, such as, but not limited to, genotypes or expression levels of genes, clinical parameters, histopathological parameters, age, gender and weight of the subject.

Upon identifying a subject as likely to develop cancer or cancer recurrence, a prophylactic procedure or therapy can be administered to the subject. For breast cancer, prophylactic measures include but are not limited to surgery (e.g. mastectomy, oophorectomy), tamoxifen administration, and raloxifene administration. For solid tumors, surgical resection can be performed.

Upon identifying a subject as having cancer or cancer recurrence, a clinical procedure or cancer therapy can be administered to the subject. For breast cancer, exemplary therapies or procedures include but are not limited to surgery, radiation therapy, chemotherapy, hormone therapy, targeted therapy, and/or administration of one or more of: Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Clafen, (Cyclophosphamide), Cyclophosphamide, Cytoxan (Cyclophosphamide), Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Neratinib Maleate, Nerlynx (Neratinib Maleate), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Ribociclib, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Vinblastine Sulfate, Xeloda (Capecitabine), and Zoladex (Goserelin Acetate).

### Kits

Also provided herein are kits for performing targeted bisulfite amplicon sequencing on a sample isolated from a subject to determine the methylation of selected genes or regions. In some aspects, the kit comprises, consists of, or consists essentially of one or more PCR primer pairs suitable for amplifying at least one region in Table 2 or 3 or a region within 10³ kb of a gene listed in Tables 1 or 3. In further aspects, the kit comprises 2, 3, 4, 5, 6, 7, 8, 9, 10, 11, 12, 13, 14, 15, 16, 17, 18, 19, 20, 21, 22, 23, 24, 25, 26, 27,28, 29, 30, 31, 32, 3, 34, 35, 36, 37, 38, 39, 40, 41, 42, 43, 44, 45, 46, 47, 48, 49, 50, 51, 52, 53, 54, 55, 56, 57, 58, 59, or 60 primer pairs directed to regions in Table 2 or 3 or within 10³ kb of the genes listed in Tables 1 or 3. In some aspects, a kit further comprises one or more reagents for bisulfite conversion and/or DNA extraction from a sample. In some aspects, the kit further comprises instructions for use.

### EXAMPLE 1

This example relates to identification of a methylation panel by whole genome bisulfate sequencing as described in Legendre et al. Clinical Epigenetics (2015) 7:100, incorporated herein by reference.

### Clinical Characteristics of Samples

The plasma methylome of MBC was characterized by paired-end whole-genome bisulfite sequencing (WGBS) to identify differentially methylated regions that were uniquely found in circulating cfDNA of a pool of 40 MBC when compared with a pool of 40 H and a pool of 40 DFS. MBC samples represented metastasis to usual sites including bone (n=23), liver (n=12), brain (n=3), lung (n=17), and soft tissue (n=6). All but five samples had involvement of more than one site. For the DFS cohort, the average years disease-free equals 9, with a range of 3-27 years. The groups were relatively matched for age at diagnosis and race. The median age for H, DFS, and MBC was 48, 42, and 42, respectively. Furthermore, the DFS and MBC groups showed comparable hormone-receptor and Her2-receptor status and prior therapy regimens (Table 4).

TABLE 4

| | DFS (for prior breast cancer) | | | MBC | | |
|---|---|---|---|---|---|---|
| | yes | no | unknown | yes | no | unknown |
| Hormone Receptor Positive (ER or PR) | 20 | 12 | 8 | 26 | 13 | 1 |
| Her2 Receptor Positive | 3 | 11 | 24 | 18 | 19 | 3 |
| Triple Negative | 6 | 20 | 14 | 5 | 26 | 9 |
| Lumpectomy | 21 | 19 | 0 | 11 | 29 | 0 |
| Mastectomy | 15 | 25 | 0 | 21 | 19 | 0 |
| Bilateral Mastectomy | 11 | 29 | 0 | 10 | 30 | 0 |
| Chemotherapy | 30 | 8 | 2 | 34 | 6 | 0 |
| Radiation Therapy | 22 | 15 | 0 | 27 | 13 | 0 |
| Hormone Therapy | 18 | 19 | 2 | 25 | 15 | 0 |
| No prior therapyz | | | | 1 | | |
| Chemotherapy for Metastasis | N/A | N/A | N/A | 33 | 16 | 1 |
| Hormone Therapy for Mets | N/A | N/A | N/A | 16 | 10 | 4 |

***Note:
These are patient self-reported data. Unknown means patient does not know.

### Summary of WGBS Statistics

For quality control assurances, cfDNA-fragment sizes were confirmed as near equal between samples pre- and post-fragmentation, and the DNA library yields and percent-alignment rates were nearly equal for the three sample pools. A total of approximately 504, 625, and 948 million reads were obtained for H, DFS, and MBC, respectively, using ten lanes of sequencing on an Illumina HiSeq 2500. Among these reads, a mean of 64.3% of reads were nonduplicated. A final read count of ˜227 (H), ˜295 (DFS), and ˜518 (MBC) million reads were used for downstream analyses. The average depth of coverage after deduplication was 7.4 (H), 9.6 (DFS), and 16.9 (MBC). The number of CpG sequenced was 28,162,972. Of these CpGs, 61.9, 74.8, and 85.7% were included in further analysis in H, DFS, and MBC, respectively. The increased coverage in MBC was not due to global copy number alterations as captured by SVDetect.

WGBS Demonstrated Global Hypomethylation and Focal Hypermethylation in cfDNA of MBC Compared with H and DFS, Which had a High Degree of Similarity

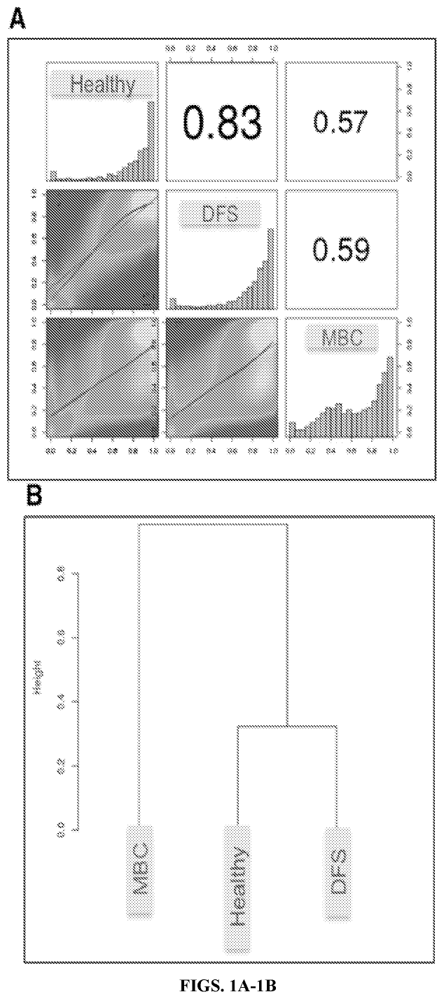

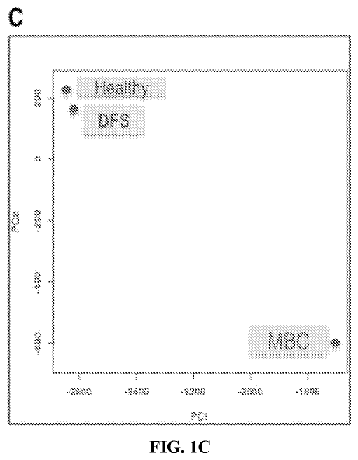

To assess the similarity of each sample group to the others, methylKit was used to compute pair-wise Pearson correlation coefficients, hierarchical clustering (Ward's method, correlation distance metric), and Principal Component Analysis (PCA) on % CpG methylation profiles. These analyses demonstrated that the H cohort closely resembled DFS, evidenced by Pearson correlation coefficient (0.83) and close proximity by hierarchical clustering and PCA (FIG. 1). However, MBC varied dramatically from H and DFS according to each analysis type, where the Pearson correlation coefficients were 0.57 and 0.59 and showed a large degree of separation by clustering and PCA. The percent methylation values per base for each sample group demonstrated that the majority of loci in DFS and H were methylated (major peak close to 1), whereas MBC had a significant proportion of loci shifted to the left indicating low methylation states and hypomethylation compared to H and DFS (FIG. 1A). To rule out a chromosomal bias, this analysis was performed for each chromosome (excluding X and Y) and confirmed a similar trend.

Identification of 21 CpG island hypermethylated hotspots in circulation of MBC

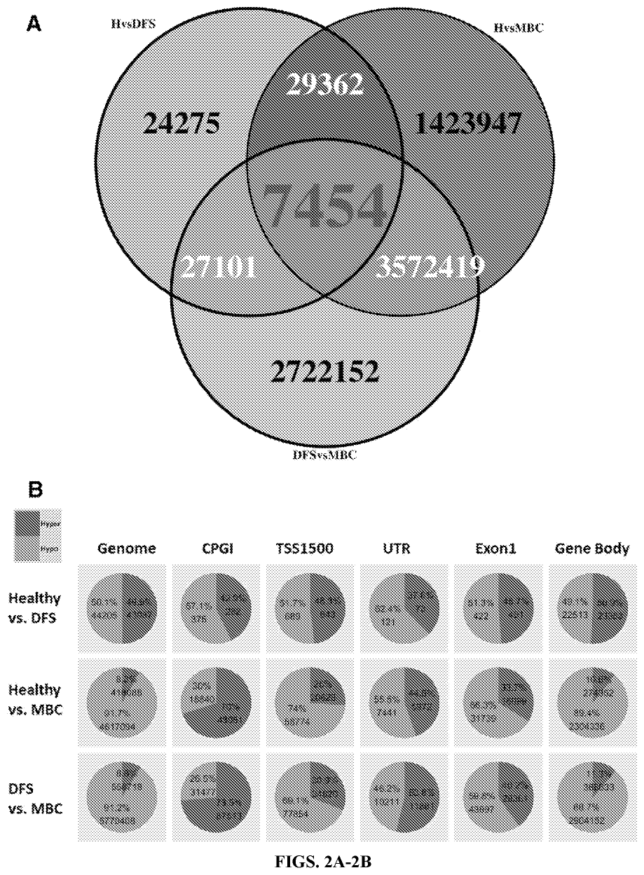

MethylKit was used to perform pair-wise differential methylation analysis at a single base-pair level. The number of differentially methylated loci (DML) between H and DFS was relatively small (n=88,192), again indicating the similarity between the groups. In contrast, ˜6.3×10⁶ DML were detected between MBC and DFS and ˜5.0×10⁶ DML detected between MBC and H (FIG. 2A). A Venn diagram (FIG. 2A) showing the overlap of DML from each comparison demonstrates a high degree of overlap when MBC is compared to either H or DFS. However, very little overlap exists with the H vs. DFS DML list when compared to the DML list generated in the two MBC comparisons. Greater than 90% of DML were hypomethylated in MBC compared with either H or DFS, indicating genome-wide global hypomethylation in the plasma of MBC (FIG. 2B). To discern the biological impact of differentially methylated loci, each event was put into a genomic context: CpG island, TSS1500, UTR, Exon 1, and Gene Body (FIG. 2B). Approximately 9% of DML were hypermethylated in MBC compared to either H or DFS. The greatest number of hypermethylated DML occurred in CPGIs (˜70%). There was also significant (P value <0.05) hypermethylation occurring in UTRs (˜50%), Exon 1 (˜35%), and TSS1500 (˜30%). Hypermethylation occurred least frequently in gene bodies (˜11%), which were predominately hypomethylated.

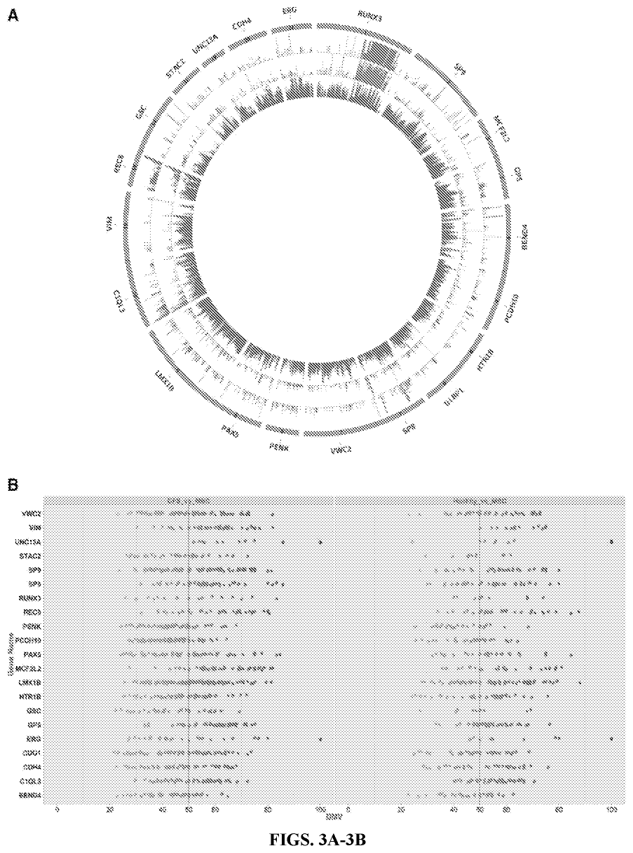

To mine the data for potential biomarkers of MBC, hypermethylated loci were focused on specifically in CPGIs because they tend to be focal in nature and were identified as the regions that differed most dramatically from normal or disease-free patterns. Regions with eight or more hypermethylated loci with differential methylation values (DMVs)>50 were specifically selected. With these criteria, 21 CPGI hotspots were identified (referred to as CpG4C), within the following genes: BEND4, CDH4, C1QL3, ERG, GP5, GSC, HTR1B, LMX1B, MCF2L2, PAX5, PCDH10, PENK, REC8, RUNX3, SP8, SP9, STAC2, ULBP1, UNC13A, VIM, VWC2 (FIG. 3).

Validation of WGBS Using Targeted Bisulfite Amplicon Sequencing with MiSeq

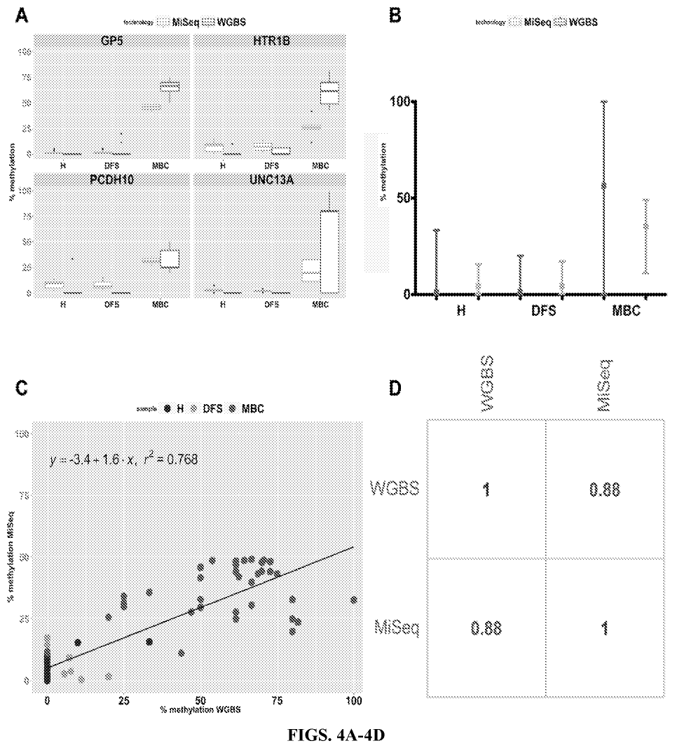

Bisulfite amplicon sequencing was performed on Illumina's MiSeq platform for technical validation of WGBS on an independent extraction of plasma from each group. This nascent, deep-sequencing strategy allows for sensitive detection of DNA methylation in low-input samples such as plasma. GP5, UNC13A, PCDH10, and HTR1B genes were selected and bisulfite PCR primers were designed within the region of interest. Each amplicon detected between 6-18 CpG loci. Targeted bisulfite amplicon sequencing on the MiSeq platform showed very good concordance with WGBS and demonstrated statistically significant (P value<0.05) increased methylation in MBC compared with H and DFS in GP5, PCDH10, HRR1B, and UNC13A (FIGS. 4A-4B). The MiSeq data also maintained that H and DFS are virtually unmethylated within these amplicons (FIGS. 4A-4B). All comparisons between MBC and H or DFS were statistically significant (P value<0.05) by Fisher's Exact Test and ANOVA, while surviving multiple test correction (q value 50.5). To further assess the degree of correlation between MiSeq and WGBS data for the amplicons containing the 36 CpG assayed, Applicant performed a scatter plot analysis and a Pearson correlation analysis to compare the 36 loci, for all groups, between the two technologies. This analysis demonstrated a high degree of correlation between MiSeq and WGBS (R2=0.768 and Pearson Correlation=0.88) (FIGS. 4C-4D). All loci in H and DFS (green and blue dots, respectively) clustered to very low methylation states to the lower left of the graph and CpG loci in MBC (red dots) mostly scattered to the upper right (FIG. 4C).

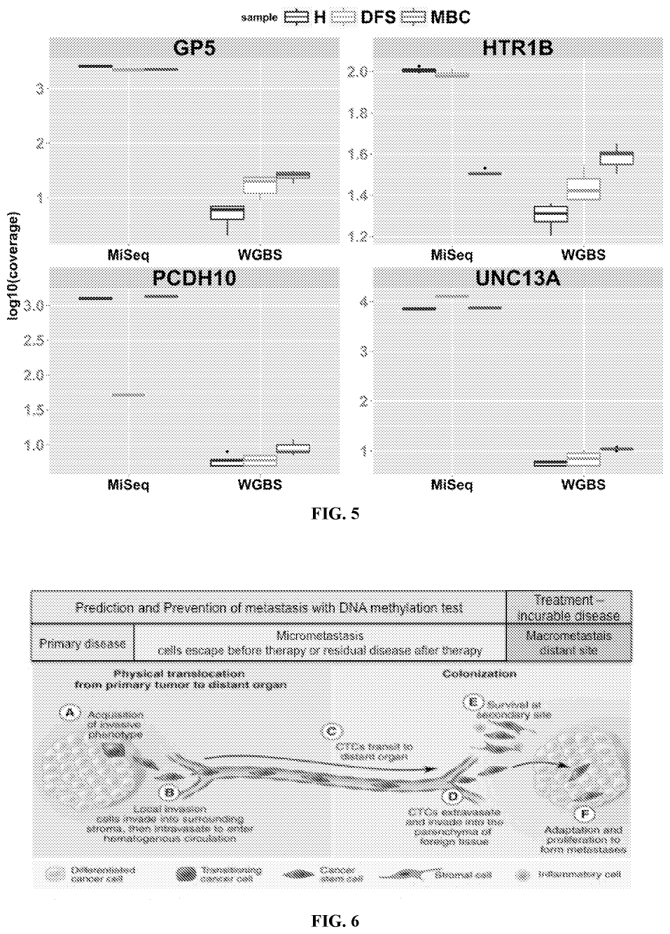

To demonstrate the expected higher coverage of MiSeq with WGBS, the mean depth of coverage for each CpG locus, within each amplicon, for each group (FIG. 5). The overall average depth of coverage for the 36 CpG loci in H, DFS, and MBC by WGBS was 10, 9.4, and 11. The average number of reads for H, DFS, and MBC by MiSeq was 3012, 2583, and 2516, respectively.

### Gene Ontology Implications for CpG4CTM

In order to demonstrate the association of the 21 gene panel to biological processes Applicant performed the Core Analysis in Ingenuity' Pathway Analysis (IPA*). The top disease implication was Cancer showing involvement of 17/21 genes. The Top Molecular and Cellular Function was Cell-Cell Signaling and Interaction. Within the Cancer disease process, 17 genes were associated with Digestive System Cancer. VIM and CDH4 were implicated in invasive cancer.

### Discussion

Cancer metastases arise from disseminated cells of the primary tumor mass before treatment and/or from minimal residual disease (MRD) persisting after therapy (collectively known as micrometastatic residual disease). Currently, there are still no effective methods to determine which patients harbor micrometastatic disease after standard breast cancer therapy and who will eventually develop local or distant recurrence. It would be advantageous to determine the subset of patients who harbor micrometastatic cells and develop trials that would evaluate the use of additional therapy for eventual prevention of metastasis. There is likely a predictive clinical window of opportunity to detect microscopic disease in the early disease setting before micrometastases lead to incurable macrometastases years after initial diagnosis.

The study described in this example represents one of the first whole-genome studies describing the plasma methylome and the first unbiased study reporting the circulating methylome of MBC, resulting in the identification of a 21-gene hotspot methylation panel that can potentially be used for prediction of metastasis in the pre-macrometastatic setting. Also novel to this study is the comparison of the plasma methylome of MBC to that of both H and DFS, making the DML hotspots highly unique to patients with clinical evidence of MBC. While other studies have reported the detection of tumor-associated DNA methylation changes in cfDNA, targets were usually selected a priori from tissue microarray data and measured using targeted approaches and not directly associated with MBC. Furthermore, genome-wide DNA methylation profiles of DFS resemble plasma methylomes from healthy individuals. This suggests that methylation patterns in cfDNA can be used to discriminate a true signal from normal-derived, background noise; the patterns may be used to detect the presence of micrometastatic residual disease after therapy. Additionally, circulating methylomic landscape of MBC is congruent with knowledge of a cancer cell's DNA methylation patterns, characterized by global genome-wide hypomethylation and focal hypermethylation, found most frequently in CPGIs. Accordingly, the data demonstrate, that the hypermethylated regions detected are regions that are generally unmethylated in the genome.

### Methods

### Sample Acquisition and DNA Extraction

120 retrospectively collected plasma samples were obtained from the Komen Tissue Bank (KTB), IU Simon Cancer Center representing 3 cohorts of 40 individuals: cohort 1 is MBC to various organs; cohort 2 is DFS (range: 3-27 years, average 9 years DFS); cohort 3 is H with no history of cancer. Samples were obtained under informed consent following Komen Tissue Bank Institutional Review Board approval. Plasma collection and processing is critical to the reproducibility of tests involving cfDNA. The KTB uses a highly standardized and meticulous protocol for processing plasma to ensure separation from blood and subsequent storage in a highly time efficient manner. Details on KTB's plasma collection SOP can be found on their website (komentissuebank.iu.edu/researchers/standard-operating-procedures/). A plasma pool for each cohort was created by mixing 50 μl of a pre-aliquoted plasma sample per individual, followed by extraction of cfDNA from 1 ml of each pool using the QIAamp DNA Micro Kit (Qiagen) according to the manufacturer's protocol, with the exception that Applicant used 1 μg of carrier RNA. DNA yields from four independent 1-ml extractions of each pool were highly consistent. The manufacturer's protocol for “Isolation of Genomic DNA from Small Volumes of Blood” was followed, with the exception that reagents were scaled up proportionally, and the sample was serially extracted on the column to accommodate the increased volume. DNA was eluted in AE Buffer (Qiagen) and quantified using the Qubit dsDNA High Sensitivity fluorometric assay (Invitrogen).

### DNA Methylation Analysis by Whole-Genome Bisulfite Sequencing

Directional bisulfite-converted libraries for paired-end sequencing were prepared using the Ovation Ultralow Methyl-Seq Library System (NuGen). The manufacturer's suggested protocol was followed. Briefly, this entailed fragmentation, end repair, adapter ligation, final repair, bisulfite conversion, and PCR amplification. 27, 14, and 33 ng of DNA were used for H, DFS, and MBC, respectively, in 50 μl T low E buffer, which was fragmented to an average size of 200 bp using the Covaris S2 system (Additional file 3: FIG. S2A). Bisulfite conversion was performed using the EpiTect Fast DNA Bisulfite Kit (Qiagen) as per manufacturer's instructions. Post-library QC was performed with BioAnalyzer DNA 1000 chips (Agilent) and the Qubit dsDNA High Sensitivity fluorometric assay (Invitrogen). An equimolar pool of the prepared libraries was created at a concentration of 5 nM. The sample was subsequently diluted and clustered on the Illumina cBot using TruSeq Paired End Cluster Kit v. 3 chemistry. Paired-end sequencing was performed on the Illumina HiSeq 2500 platform using TruSeq SBS v3 kits for a total read length of 200 bp.

### Targeted Bisulfite Amplicon Sequencing

Targeted bisulfite amplicon sequencing was performed on the MiSeq (Illumina) using an independent replicate of the three plasma pools for validation of CpG island hotspots for GPS, HTR1B, PCDH10, UNC13A. Bisulfite Primer Seeker 12S (Zymo Research) was used to create primer-pairs specific for bisulfite-converted DNA, which produced PCR amplicons ranging in size from 109-235 base pairs. The bisulfite conversion was accomplished using EZ DNA Methylation-Gold Kit (Zymo Research) according to the manufacturer's standard protocol. Forty cycle PCR reactions were carried out with the Zymo Taq (Zymo Research) kit and the manufacturer's recom mended conditions using 2μl of converted DNA template per 30₁1.1 reaction. Reactions were purified using NucleoSpin columns (Macherey-Nagel) as per the manufacturer's suggested protocol. Purified reaction products were run out on a 2% agarose gel for visual inspection and quantified using the Qubit dsDNA High Sensitivity fluorometric assay (Invitrogen).

A 266-ng equimolar mix of the four amplicons was used as input for sequencing library preparation using the Kapa Hyper Prep Kit (Kapa Biosystems). TruSeq DNA LT adapters (Illumina) were used for indexing. No post-ligation amplification was performed. Quantitative-PCR library quantification was carried out using the Kapa Library Quantification Kit (Kapa Biosystems).

Equimolar library pools were created and diluted to 15 pM for denaturation. PhiX Control v3 (Illumina) was spiked in at a 5.0% final concentration, and subsequent cluster generation/sequencing was performed on the MiSeq using MiSeq Reagent Nano Kits (Illumina). Five hundred cycles of 2×250 paired-end sequencing generated over 820,000 reads.

### Data Processing and Analysis

Bisulfite-modified DNA reads from WGBS and MiSeq were aligned to the bowtie2-indexed reference genome GRCh37-62 using Bismark tool version 0.12.7. Bismark relies on two external tools, bowtie (bowtie-bio.-sourceforge.net/index.shtml) and Samtools (www.htslib.org). bowtie2 version 2.0.0-beta6, and Samtools version 0.1.19 were used. Bismark was used as suggested except for the bowtie2's parameter N (number of mismatches in a seed alignment during multispeed alignment) where the value of 1 was used for increased sensitivity. Next, PCR duplicates were removed for WGBS using default parameters. Methylation calling was also processed using a Bismark module called “Methylation Extractor,” which was used according to the author's specifications. Base-pair level differential methylation analysis was implemented using the R package methylKit 0.9.2. Bismark's sam file output was used as input to methylKit and data imported using the embedded function “read.bismark”. The minimum read coverage to call a methylation status for a base was set to 5, and the minimum phred quality score to call a methylation was set to 20. The read.context option was set to “CpG”. Other options to the read.bismark function were set to default values. The following pair-wise comparisons were performed in methylKit using the Fisher Exact Test: H versus DFS, H versus MBC, and DFS versus MBC for both WGBS and MiSeq datasets. Before calling differential methylation, each comparison was methylKit-reorganized, united, and then underwent differential methylation analysis using methylKit functions. With a minimum of five reads in each group, a differential methylation value (DMV) of 20 (in percent scale) and P values<0.05 were considered DML. For WGBS and MiSeq, chromosome X and Y reads were removed. MethylKit DML calls were annotated according to genomic location: Exon 1, Gene Body, TSS1500, UTRS-prime, and CPGI annotations. For selection of biomarkers, Applicant identified CPGIs with at least 8 DML having DMVs greater than 50. All loci of interest were visually inspected in Integrated Genomic Viewer (IGV).

### Abbreviations

cfDNA: cell-free DNA; CPGI: CpG island; DFS: disease-free survivors; DML: differentially methylated loci; DMV: differential methylation value; H: healthy individuals; IGV: Integrated Genomic Viewer; KTB: Komen Tissue Bank; MBC: metastatic breast cancer; MRD: minimal residual disease; WGBS: whole-genome bisulfite sequencing.

### EXAMPLE 2

This example describes additional analysis of the experiments in Example 1 and other approaches to identification of molecular profiles.

Molecular profiles have improved clinicians' ability to determine the need of chemotherapy for those individuals who are at high-risk for recurrence. The most widely used multigene predictive classifiers include the 21-gene Oncotype Dx signature (Genomic Health, USA), the 70-gene MammaPrint signature (Agendia, Netherlands), the 76-gene Rotterdam signature and the PAM50 intrinsic classifier (NanoString, USA). Despite the huge quantity of information gleaned from these gene signatures, none can precisely predict the clinical course of an individual and rely on the presence of tissue at a single time point. What all these tests have in common is they estimate the risk of harboring micrometastatic disease at the time of diagnosis which is based on the patient's tumor biology, and therefore who will benefit from systemic chemotherapy. All of these tests are only relevant for patients with ER positive tumors and are not recommended for patients with any of the other subtypes. Therefore, predictive/prognostic tests for the other subtypes are not available. More importantly, these molecular tests are very poor predictors of cancer recurrence even after appropriate surgical and medical therapy. Not unlike the clinicopathologic features, there are patients deemed high-risk who do very well with standard therapy and never experience a recurrence and patients with low-risk profiles who still die of breast cancer. There also remains a risk of recurrence in high-risk patients even after treating them with the most effective chemotherapy agents. Another strategy for stratifying patients as high-risk for systemic recurrence is pathologic status after neoadjuvant systemic therapy. However, recent meta-analyses have not demonstrated a correlation of pathologic complete response (pCR) to treatment with disease-free survival or overall survival indicating that more sensitive measurements are needed to assess response to neoadjuvant therapy in order to prognosticate outcome.

Cancer metastases arise from disseminated cells of the primary tumor mass before treatment and/or from minimal residual disease (MRD) persisting after therapy (collectively known as micrometastatic disease) (see FIG. 6 for depiction of metastatic cascade). Currently there are still no effective methods to determine which patients harbor micrometastatic disease after standard cancer therapy (e.g., breast cancer therapy) and who will eventually develop local or distant recurrence. It would be advantageous to determine the subset of patients who harbor micrometastatic cells and develop further clinical trials, to evaluate additional therapy for the eradication of residual micrometastatic disease. Without being bound by theory, Applicant believes there is a clinical window of opportunity to detect microscopic disease in the pre-macrometastatic setting before micrometastases lead to incurable macrometastases years after initial diagnosis (FIG. 6). Without being bound by theory, Applicant proposes these results are of major significance as they seek to build upon a roadmap to improving treatment strategy as well as preventing recurrence in all subtypes of cancer, e.g., breast cancer. Applicant proposes to validate a blood-based DNA methylation signature of MBC as a prognostic marker of distant and late disease recurrence in the pre-metastatic setting. This test has the strong potential of being prognostic of who is likely to develop recurrences. In addition, this test can also be developed as an end of therapy (surgical and medical) predictive biomarker for patients who would benefit from surgery after neoadjuvant therapy, and/or additional chemotherapy and surveillance. Such a marker is a major advance and acts as an adjunct to the molecular tests already on the market such as Mammaprint and OncotypeDX.

Human blood is easily accessible for sampling and contains informational cues from tumors, which “leak” protein and DNA into circulation. In the last few years, circulating cell-free (cf)DNA has attracted attention for clinical use in the context of risk prediction, prognostication and prediction of response to chemotherapy in human cancer. Early reports suggesting that the simple presence or absence of cfDNA itself, or its concentration was diagnostic have been scrutinized, since high levels of cfDNA are not specific to neoplastic lesions and are also observed in several other pathologies, including pro-inflammatory and neurological disorders. In addition, cfDNA has also been found in healthy individuals in the same concentration range of some cancer patients. This argues that the presence of tumor-specific alterations is the best criterion to assess the tumoral origin of cfDNA. Various types of DNA alterations have been reported in cfDNA including, point mutations, microsatellite instabilities, loss of heterozygosity and DNA methylation. DNA methylation is a centrally important modification for the maintenance of large genomes. The essentiality of proper DNA methylation maintenance is highlighted in cancer, where normal patterns are lost. Aberrant DNA methylation is among the earliest and most chemically stable molecular alterations in cancer, making it a potentially useful biomarker for early detection or risk prediction. The high degree of detection sensitivity of aberrantly methylated loci is afforded by the frequency of the occurrence (for example, compared to somatic mutations) and because bisulfite modification provides detection of hypermethylated targets in large excess of unmethylated ones (1:1000). Another advantage to developing DNA methylation biomarkers is that methylation values are measured as continuous variables and can incorporate measurements from multiple CpG loci. These properties of DNA methylation measurements enable monitoring of the signal over time and signal amplification—thus increasing sensitivity. No studies have reported on using this approach for prediction of metastasis in the early stage setting. Methylated RASSF1A and APC, identified in serum DNA from patients with breast cancer, were associated with a worse outcome. RASSF1A, RARbeta2, NEUROD1 were shown to be useful for monitoring the efficacy of adjuvant therapy or surgery in patients with breast cancer and another study reported a 10-gene panel associated with metastatic breast cancer. Without being bound by theory, Applicant believes there is strong rationale for using cfDNA methylation as a biomarker approach for disease prognosis and predicting recurrence in early stage breast cancer patients. Aberrant CpG island hypermethylation rarely occur in non-neoplastic and normally differentiated cells. Therefore, the DNA released from tumor cells can be detected with a notable degree of sensitivity, even in the presence of excess of DNA from normal cells and this represents a remarkable potential for clinical application.

A reproducible blood-based test for hypermethylated genes that can be used for prediction of residual microscopic disease after standard surgical and systemic therapy has yet to be successfully developed. Discovery of new markers, as well as improvements in existing technologies, are needed to provide more robust, reproducible, quantitative, sensitive, and specific assays. This Example 2 expands upon Example 1 and a published study (Legendre et al.) utilizing whole genome bisulfite sequencing (WGBS) to describe the methylome of circulating DNA in three cohorts of healthy, disease-free survivors (DFS) and MBC subjects and which lead to the identification of a 21-gene methylation signature uniquely associated with MBC. Applicant has also developed a targeted bisulfite next-generation sequencing strategy coupled with PCR multiplexing that can be used to detect DNA methylation in low input samples such as plasma and, Applicant devised a strategy permitting further analysis and validation of the methylation signature in vivo using patient-derived xenografts (PDX) of breast cancer. Without being bound by theory, Applicant's hypothesis is that a multi-gene DNA hypermethylation signature involving rationally selected hotspots detectable in circulation can be used to detect micrometastatic disease and serve as a prognostic and future predictive marker for MBC. Specifically, unlike the current molecular tests used to aid in the treatment of breast cancer which just predict which patients may harbor micrometastatic disease, this approach will also try to identify those patients that still harbor micrometastatic disease after appropriate therapy. Applicant anticipates that such a blood test would be advantageous at several time points in the treatment of newly diagnosed breast cancer: after surgery alone, after surgery and systemic chemotherapy, and after neoadjuvant systemic therapy and surgery to predict response to therapy and which patients may benefit from additional systemic therapy. This is especially important in an era where immunotherapy has been shown to be effective in many tumor types including breast cancer and could prove an important adjunct in this type of high-risk patient. Additionally, such a test could ultimately signify those patients who might be spared from unnecessary treatment. It is important to note that the goal of such a biomarker is not intended to detect macrometastasis (full-blown, clinically evident metastasis), as that is expected not to improve outcomes with current therapies—but rather to determine if a patient is at high risk of recurrence during early stage settings so that additional therapies can be developed and administered in order to prevent cancer recurrence.

Determine the Utility of a DNA Methylation Signature as a Prognostic Maker of Recurrence by Measuring the Frequency, Sensitivity and Specificity of the Marker with bAmplicon-seq

Applicant's research to date has demonstrated CpG Island (CGI) hypermethylation of 21 CGI hotspots in the circulation of MBC. This signature was identified using three pooled samples of cfDNA containing 40 different patient plasmas per pool. Therefore, in this example, Applicant validates the 21 CGI panel in the 120 individual samples used to generate the pooled WGBS from the three study cohorts and in an independent cohort of 60 MBC and 60 healthy plasma samples. In this example, Applicant 1) Determines and compares the frequency CGI methylation in cfDNA of MBC, DFS and healthy plasma; 2) Evaluates the sensitivity and specificity of 21 CGI hotspots to discriminate between MBC, DFS and H to develop a prognostic test.

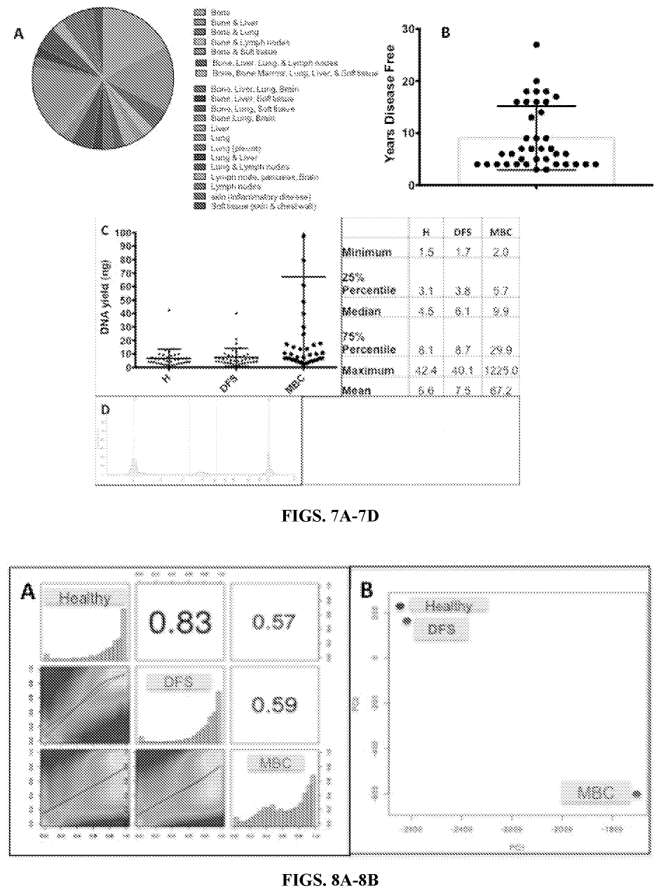

Applicant performed WGBS on cfDNA obtained from plasma samples representing 3 cohorts of 40 individuals each: cohort 1 was from MBC to various organs (FIG. 7A); cohort 2 was from DFS (FIG. 7B, range: 3 years-27 years, average 9 years DFS); cohort 3 was from healthy females with no history of cancer. MBC and DFS samples were nearly equally distributed for molecular subtype and previous therapies. About two thirds of DFS and MBC samples were ER+ and ˜20% were triple negative breast cancer. Nearly 50% of MBC and 20% of DFS samples were Her2+. The vast majority of patients from DFS and MBC groups had prior surgery and/or chemotherapy and nearly half from each group had previous radiation therapy. Lastly, over ⅔ of samples across groups were from Caucasian women with the remaining coming from African American, Asian or Hispanic women. The median age for MBC, DFS and H was 42, 42 and 48, respectively. These plasma samples were collected from the Komen Tissue Bank (KTB), IU Simon Cancer Center. Plasma collection and processing is critical to the reproducibility of tests involving cfDNA. The KTB uses a highly standardized and meticulous protocol for processing plasma to ensure separation from blood and subsequent storage in a highly time efficient manner. Details on KTB's plasma collection SOP can be found on their website (komentissuebank.iu.edu/researchers/standard-operating-procedures/). For WGBS analysis Applicant created a plasma pool for each cohort by mixing 50 μl of a pre-aliquoted plasma sample followed by extraction of cfDNA using the QIAamp DNA Micro Kit. DNA yields from three independent extractions of each pool were highly consistent. During DNA extraction from minute samples, it was paramount to avoid DNA loss during the several manipulation steps. Therefore, Applicant optimized the extraction of DNA from plasma by comparing the performance of different kits and volumes of input plasma. Accordingly, Applicant has since switched to using the bead-based MagMAY™ Nucleic Acid Isolation Kit, which Applicant used for the extraction cfDNA from 750 μl of each individual plasma sample from the three cohorts (FIG. 7C). Cell-free DNA was quantitated with the Tapestation (Agilent Technologies). Integration across the region of 130-300 bp was utilized for accurate determination of yield. The calculation encompasses the major observed peak for cfDNA at approximately 170 bp and excludes from yield calculations contaminating, high molecular weight DNA (FIG. 7D). Every single sample yielded cfDNA with yields ranging from 1.5 ng to 1225 ng (FIG. 7D). The preferred source of cfDNA is from plasma not serum. This comes from a consensus that serum contains much higher amounts of cfDNA than plasma, which is thought to be released by lysis of lymphocytes during clotting that takes place after the sample is taken from the patient. Blood serum is blood plasma without clotting factors. Libraries using 15 ng of each cfDNA pool were prepared with the Ovation® Ultralow Methyl-seq Library kit (Nugen). An equimolar pool of the prepared libraries was created at a concentration of 5 nM. The sample was subsequently diluted and clustered on the Illumina cBot using TruSeq Paired End Cluster Kit v. 3 chemistry. Paired end sequencing was performed on the Illumina HiSeq 2500 platform using TruSeq SBS v3 kits, for a total read length of 200 bp. WGBS reads were aligned to the local database using open source Bismark Bisulfite Read Mapper with the Bowtie2 alignment algorithm. QC on the data was assessed, and data analysis was conducted using the R package methylKit to identify DNA methylation differences between each cohort. Differential methylation values (DMV)>|20| and Fisher's exact test p values<0.05 were cut-offs for calling differentially methylated loci (DML). Loci on sex chromosomes were removed.

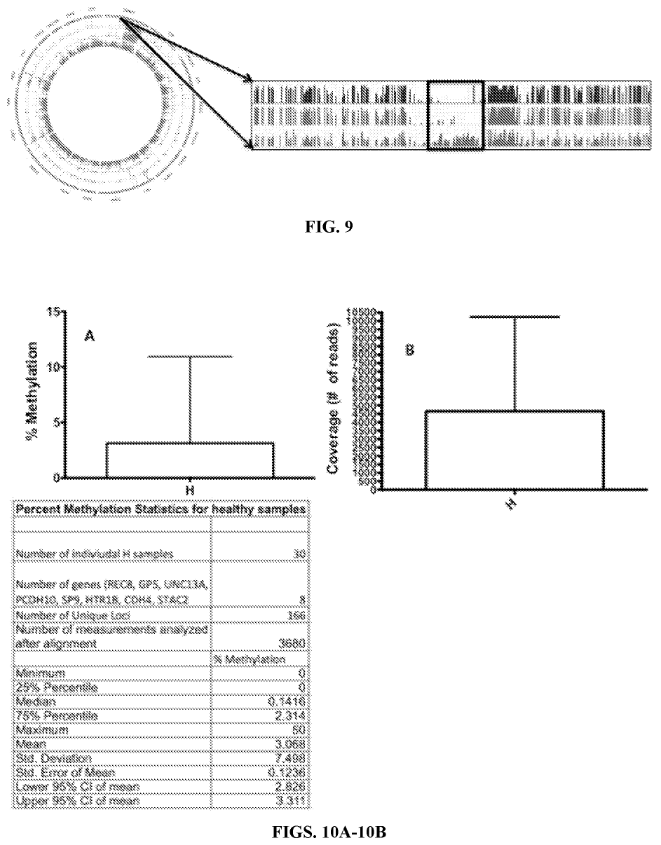

Differential methylation analysis on WGBS data demonstrated that there were relatively few differences seen between H and DFS as indicated by relatively few differentially methylated loci (n=87,935), a high Pearson correlation coefficient (0.83), hierarchical clustering and principal component analysis (FIG. 3). In contrast, approximately 5.0×10⁶ DML were detected between MBC and H or DFS. This suggests that methylation patterns in cell-free plasma DNA may be used to monitor treatment and detect the presence of residual disease. Based on these comparisons, the circulating methylomic landscape of MBC was congruent with our knowledge of a cancer cell's DNA methylation patterns, characterized by global genome-wide hypomethylation and focal hypermethylation, found mostly in CGIs (CGI). To identify putative biomarkers in MBC, Applicant selected DML with DMVs ≥50 in regions with 5 or more hypermethylated loci and where methylation in DFS and Healthy demonstrated percent methylation values less than 20 in the regions of interest. Applicant selected hypermethylated loci over hypomethylated loci because bisulfite conversion can detect hypermethylated targets in large excess of unmethylated ones (1:1000). Based on these criteria, Applicant previously identified the following 21 hotspots within CGIs of the following genes: BEND4, CDH4, C1QL3, ERG, GPS, GSC, HTR1B, LMX1B, MCF2L2, PENK, REC8, RUNX3, PAXS, PCDH10, SP8, SP9, STAC2, ULBP1, UNC13A, VIM, VWC2. (FIG. 9).

### Validation of WGBS Using Targeted Bisulfite Amplicon Sequencing

Applicant optimized bisulfite amplicon sequencing (bAmplicon-seq) for targeted methylation analysis by coupling PCR multiplexing with next generation sequencing on the MiSeq (Illumina) System. This nascent, deep-sequencing strategy allows sensitive detection of DNA methylation in low input samples such as plasma. For technical validation, an independent pool of the three plasma samples of GGI hotspots of GPS, HTR1B, PCDH10, UNC13A—was randomly selected from the 21 genes. Briefly, bisulfite Primer Seeker 12S (Zymo Research) was used to create primer-pairs specific for bisulfite converted DNA, which produced PCR amplicons containing 6-18 CpG loci and PCR reactions were multiplexed. Bisulfite conversion was accomplished using EZ DNA Methylation-Gold Kit (Zymo Research) according to the manufacturer's standard protocol. A 266 ng equimolar mix of the four amplicons was used as input for library preparation using the Kapa Hyper Prep Kit (Kapa Biosystems). TruSeq DNA LT adapters (Illumina) were used for indexing. No post-ligation amplification was performed. Equimolar library pools were created and diluted to 15 pM for denaturation. PhiX Control v3 (Illumina) was spiked in at a 5.0% final concentration and subsequent cluster generation/sequencing was performed on the MiSeq using MiSeq Reagent Nano Kits (Illumina). Five hundred cycles of 2×250 paired-end sequencing generated over 820,000 reads. Bisulfite-modified DNA reads for MiSeq were aligned and analyzed as described for WGBS.

Targeted bisulfite amplicon sequencing on the MiSeq platform showed very good concordance with WGBS, and demonstrated statistically significant (p-value<0.05) increased methylation in MBC compared with H and DFS in GPS, PCDH10, HRR1B and UNC13A. The MiSeq data also maintained that H and DFS are virtually unmethylated within these amplicons. All comparisons between MBC and H or DFS were statistically significant (p-value<0.05) by Fisher's Exact Test, while surviving multiple test correction (adjusted p<0.05). These data suggest that the frequency of lowly methylated controls and highly methylated MBC is moderate to high. To further assess the degree of correlation between MiSeq and WGBS data for the amplicons containing the 36 CpGs assayed, a scatter plot analysis and a Pearson correlation analysis was performed to compare the 36 loci, for all groups, between the two technologies. This analysis demonstrated a high degree of correlation between MiSeq and WGBS data (Pearson Correlation=0.88). All loci in H and DFS (green and blue dots respectively) clustered to the lower left of the graph and CpG loci in MBC (red dots) mostly scattered to the upper right. To demonstrate the expected higher coverage of MiSeq with WGBS, the mean depth of coverage for each CpG locus was calculated, within each amplicon, for each group. The overall average depth of coverage for the 36 CpG loci in H, DFS and MBC by WGBS was 10, 9.4 and 11. The average number of reads for H, DFS and MBC by MiSeq was 3012, 2583 and 2516, respectively. Therefore, it is expected that targeted bisulfite sequencing will enable the requisite sensitivity for future clinical development of a biomarker that can detect micrometastasis and indicate high-risk breast cancer patients.

To explore the potential sensitivity of the markers in individual samples, the expected number of positive samples was computed using the % methylation estimates from pooled DNA. No more than 4 individual patients from H or DFS pools can be more than 20% methylated if the estimate in the pool is 2% suggesting multiple CpG loci with specificity>90%. At the same time, a minimum of 10 individuals from MBC are more than 5% methylated for a 25% frequency in pooled DNA, and at least 20 individuals are highly methylated for frequencies of 50%. Without being bound by theory, it is believed that signal from hundreds of CpGs can be combined in order to build a sensitive classifier for MBC detection. As further evidence, individual samples were analyzed and demonstrated that 30 healthy samples maintained extremely low levels of cfDNA methylation for 8 of the target regions analyzed by bAmplicon-seq (FIG. 10). Greater than 68% of 3680 total CpG measurements had % methylation <2% and 80% were less than <5% methylated. This data supports the hypothesis that the cfDNA methylation biomarker can have high sensitivity and discriminate methylation signals from MBC cells even against the normal cfDNA background in each sample.

### Determine the Frequency, Sensitivity and Specificity of a 21 Gene DNA Methylation Circulating Signature as a Prognostic Test in Retrospectively Collected Plasma Samples

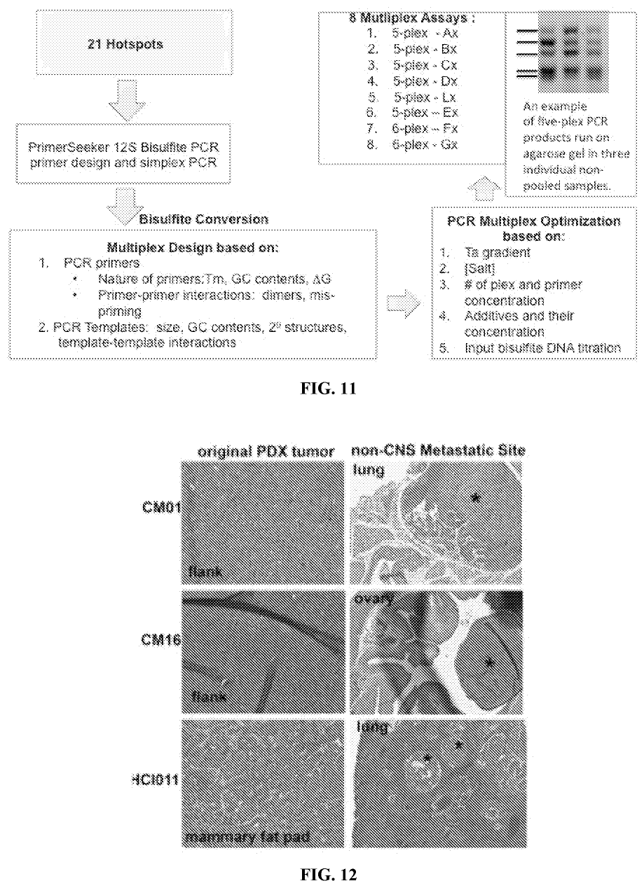

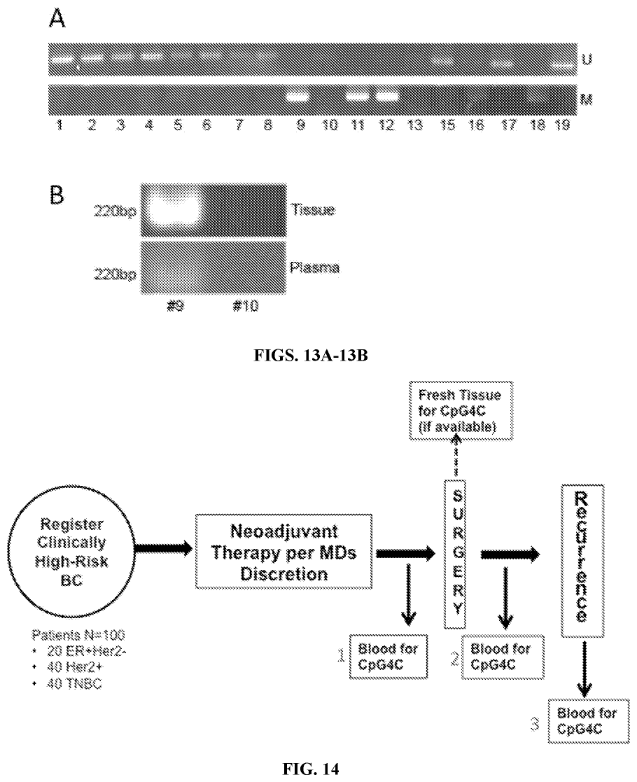

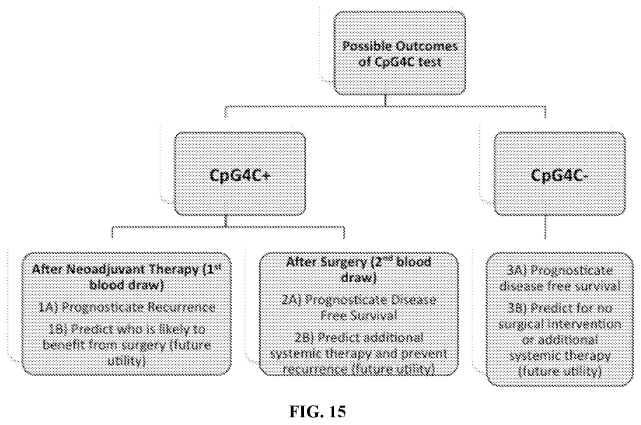

In this example, each individual plasma sample obtained from the KTB is analyzed to calculate the frequency of samples with methylation across the CGI hotspots and to determine the sensitivity and specificity of the 21 CGI hotspots to discriminate MBC from H and DFS. A total of 42 simplex PCR assays (2 individual assays per hotspot/region of interest, 504 total CpGs) were designed, and 8 separate multiplex assays were optimized for bAmplicon-seq on the MiSeq system (FIG. 11). Bisulfite PCR and multiplexing conditions were optimized for a variety of variables and the workflow implemented as described above and as presented in FIG. 11.

Validate CpG4C in an Independent Cohort of Plasma Samples from Healthy and MBC Samples.

In this example, additional plasma samples are analyzed from women with MBC and healthy women to determine the sensitivity and specificity of the CpG4C test to discriminate MBC. The demographics of the additional MBC samples were selected to be similar to that of the original 40 MBC samples from KTB. These additional samples have been purchased from Conversant Bio—a commercial vendor. Conversant Bio uses a highly standardized and meticulous protocol for processing plasma to ensure separation from blood and subsequent storage in a highly time efficient manner. cfDNA is extracted as described above from each individual plasma sample using the MagMAX™ Nucleic Acid Isolation Kit and bisulfite amplicon sequencing performed for CpG4C.

Sequence data is processed using the pipeline described above and for each CpG site, DNA methylation level estimated as the fraction of methylated reads. Each hotspot is summarized by two bAmplicons and each bAmplicon will cover from 6-18 CpGs. To evaluate each of the 21 CGI hotspots, biomarker signatures of MBC are constructed using stability selection with elastic-net regularized logistic regression. The individual CpG sites from all identified CGI hotspots are included in a regularized logistic model with the outcome variable indicating MBC verses H or DFS. The elastic-net penalty (1) allows for correlation in cytosine methylation for neighboring CpG sites as DNA methylation in CpG islands is often correlated for distances<200 bps and (2) results in a model including only those CpG loci that are the most significantly associated with MBC. Others have published predictive signatures in cancer using this approach. The final model are referred to as the CpG4C (4C=foresee) test and are validated in an independent set of 60 MBC and 60 healthy plasma samples. The final model results in a probability estimate for a sample being MBC and be analyzed using receiver operator characteristic (ROC) analysis. The true-positive rate (TPR) and the false-positive rate (FPR) are measures of biomarker performance. Also known as the sensitivity, the TPR is the proportion of diseased people correctly detected as having disease by use of the marker. The FPR (1—specificity) is the proportion of control cases incorrectly detected as having disease by use of the marker. The ROC curve is a graph of sensitivity (TPR on y-axis) versus 1-specificity (FPR, x-axis). To evaluate the utility of the CpG4C final model Applicant constructs a ROC curve and compute the area under the curve (AUC) to assess the best cut-off in regards to the specificity and sensitivity of the model. A model with AUC value in excess of 0.8 would indicate high specificity and sensitivity. With 40 samples in each group (H, DFS and MBC), and assuming a uniform [0,1] distribution for AUC, the power to detect an AUC of 0.8 versus an AUC of 0.5 is at 95% at the 0.05 significance level. Applicant identifies the cutoff for a 10% FPR and determine the sensitivity for any larger test value correctly identifying MBC. In example ii) Applicant reports the frequency of subjects testing positive for this cut-off in an independent set of 120 samples (60 MBC/60 H). We power the independent test set to exceed a minimum TPR of 60% for a maximum FPR of 10%. With 60 samples in each group, the power is 82% to validate a test with 85% TPR at 10% FPR (0.05 significance level). Applicant utilizes frequency table analysis and Chi-square tests to assess the association of ER and Her2 status and distant site of recurrence with CpG4C (dichotomized using the cut-off value) in the MBC group. As within the DFS cohort there are 2 clusters of survivors (FIG. 7B), one cluster with a DFS range from 3-10 years and the second cluster with a range of survivors from 13-27 years, Applicant also looks for associations of DFS sub-groups with CpG4C. Lastly, Applicant re-computes from the combined set of 140 H and DFS samples the cut-off for a 10% FPR to carry forward in the examples below.

The goal of this example is to determine the frequency, sensitivity, specificity and subtype association of a CGI methylation panel in individual plasma samples of MBC, DFS, and healthy individuals. Without being bound by theory, Applicant expects that the regularized logistic regression model will result in a highly specific and sensitive model, referred to as a CpG4C test and which can be further developed as a prognostic or predictive biomarker of recurrence.

### Determine the Analytical Limit of Detection and Track How Different Degrees of Tumor Burden Impacts Mythylation Status of CpG4C in Preclinical Models of Breast Cancer Metastasis

The goal of CpG4C is to identify women with early stage breast cancer who remain at high risk of recurrence upon completion of therapy. The 21-gene signature was derived from women with MBC at the time blood was drawn. At this point the methylation differential with control subjects is large and the tumor burden is high. The present example is directed toward developing a biomarker that can be used for prognostication (and future prediction) of recurrence at the end of therapy in women with early stage breast cancer. At this point in patient care, the tumor burden is significantly lower and any remaining disease is subclinical making the methylation differential expectantly lower than women with full-blown disease burden. Therefore, a biomarker test will need to be highly sensitive to be used at the end of therapy time-point of patient care. DNA methylation detection has the potential to meet these requirements because the methylation value is a continuous variable ranging from 0-100 (not binary—on or off) and because the signal is coming from numerous CpG loci. For example, a single point mutation is either there or not there. However, for CpG methylation there is plenty of opportunity to detect signal and there is a dynamic range of detection. Also, since the background is expected to be low in healthy controls (FIG. 10) means high signal to noise ratios and a greater chance to detect small changes in methylation. Furthermore, using deep-targeted bisulfite sequencing (in the order of >2000×) improves the ability to detect small changes in DNA methylation but questions remain to be answered. Therefore, the purpose of this experiment is to better determine the analytical limit of detection of CpG4C using bAmplicon-seq and to determine the effect that changing degrees of tumor burden has on detection of differentially methylated regions in cfDNA. Additionally, the correlation of tissue DNA methylation with cfDNA from the same mice is examined. This experiment has far-reaching implications beyond CpG4C and are quite informative as to the nature of cfDNA methylation detection in circulation.

The use of PDX models to measure cfDNA methylation is highly novel. Applicant has performed proof of concept and feasibility studies to demonstrate that plasma can be harvested to isolate cfDNA from mouse blood and perform DNA methylation analysis. Applicant has a rich resource of PDX models including a series of 5 PDX models derived from patients with breast cancer brain metastasis (FIG. 12, Table 1). Also obtained are 18 PDXs derived from women with aggressive breast cancer. Collectively, the models represent Her2+, ER+ and triple negative breast cancer, are clinically annotated and very well molecularly characterized. Furthermore, the PDXs tended to recapitulate the human form of the disease. Some models form metastases in mice in manner similar to the patient's history and other models from brain metastasis also continue to show evidence of metastasis in mice similar to other metastases seen in the patient (FIG. 12, Table 1). Both tissue and plasma have been harvested from the 23 PDX tumors and DNA and cfDNA has been extracted, respectively.

For plasma isolation, matching whole blood (up to 200 μl per animal) was collected into heparinized tubes from 3-5 mice per PDX by pricking the submandibular vein with a sterile disposable lancet. This yields approximately 100 μl of plasma per mouse. Blood was also from non-tumor bearing animals for control purposes. Blood was processed immediately for isolating plasma according to SOPs. On average, approximately 3-5 ng of cfDNA was recovered from non-tumor bearing mice and 5-10 ng from tumor bearing mice, which when totaled between all three biological replicates is sufficient for subsequent assays.

Applicant has assessed both the analytical limit of detection and the correlation of tissue DNA methylation with cfDNA methylation from the same mouse. For this, methylation specific PCR (MSP) for the RUNX3 hotspot region was performed. It was first confirmed that RUNX3 hotspot shares little homology to the mouse gene. Briefly, DNA was bisulfite treated and MSP performed using standard conditions and previously published primer pairs that detect methylated (M) or unmethylated (U) bisulfite DNA. Next, RUNX3 MSP was performed on 18 PDX tumor DNAs to identify methylation positive and negative PDXs. RUNX3 was hypermethylated (M +, U-) in 3 PDXs with Luminal B disease (#s 11,12,18) and 2 PDXs with triple negative breast cancer (#s 9&16), which all had known metastatic potential in vivo and it was unmethylated (U+, M−) in 13 PDXs with and without metastatic potential (FIG. 11). Next, the cfDNA from one M+ tumor and one U+ tumor was tested a correlation was confirmed between tissue and plasma in these samples.

The limit of detection of cfDNA methylation was assessed by spiking increasing amounts (0-10 ng) of artificially methylated human genomic DNA in to 2 independent 100 μL aliquots of mouse plasma from non-tumor bearing NOG mice. CfDNA was extracted and MSP was performed. Human RUNX3 methylation was detected with as little as 0.01 ng, but reproducibly with 1 ng of human spike in (sensitivity higher by bAmplicon-seq). No methylation was visible in the unspiked control.

### Conduct Analytical Validation Experiments to Determine the Limit of Detection of CpG4C Methylation in Plasma

These proof of concept and feasibility studies have paved the way for the experiments outlined herein. First, Applicant expands on the limit of detection studies by spiking in human methylated DNA in 10 fold increments (0.001-10 ng) into plasma from non-tumor bearing NOG mice collected as described above (this strain are used for all subsequent studies) and from healthy humans (purchased from Conversant Bio). Unspiked samples are used as controls. Since the commercially available human genomic DNA is high molecular weight Applicant first shears the DNA down to the size of cfDNA (167 bp) using a focused ultrasonicator (Covaris) before spiking in to 500 ul of plasma. DNA is then extracted from triplicate 500 μl aliquots of plasma and quantitated as described in this example. Samples from this 6 point spike-in undergo bisulfite conversion, multi-plex PCR amplification using the multiplex PCR assays already developed, undergo PCR clean-up, library preparation and sequenced to >2000× on the MiSeq. Data from sequencing are analyzed as described in preliminary data in this example. For spike-in of mouse plasma, Xenome (an algorithm used to determine species sequence identity) are used to align only human data. The percent methylation and depth of coverage are calculated for each sample and each locus. The coefficient of variation (CV) are calculated for biological replicates. The detection limit are determined by plotting the distribution of percent methylation values for the unspiked controls and the spiked controls at different input amounts. The limit of detection are calculated as the lowest quantity that can be distinguished from the unspiked control within a 90% confidence limit. Performing the experiment in triplicate ensures that the 90% lower confidence bound for the 5% methylation fraction spike-in exceeds 2% for coefficients of variation of 0.8 and smaller.

Determine Impact of Tumor Burden on CpG4C Methylation Detection in PDX Models of Breast Cancer Metastasis from cfDNA and Determine the Correlation of Tissue/Plasma DNA.

The degree of tumor burden impacting overall signal is also related to the detection limit. However, the difference is that the first example is an empirical and analytical validation of detection limit whereas the second example deals more with the biological impact on detection. First, the cfDNA and tumor DNA already extracted from the series of 23 PDX tissues and plasmas is used to determine CpG4C methylation by targeted bAmplicon-seq as described in this example. Applicant compares tissue to plasma methylation levels from matched mouse by performing Pearson Correlation analysis for each CpG position queried by the assay. Pearson correlation coefficients>0.8 are considered sites well correlated. From this series, Applicant selects 5 PDX models positive for CpG4C in cfDNA to assess the sensitivity of the test as a function of tumor burden. Applicant tests 24 animals per model, requiring a total 120 animals as described below.

The 5 selected PDXs are thawed from cryopreservation and implanted into mammary fat pads of 5 6-week-old severely immunodeficient NOG female mice. Due to the scope of work, one model is analyzed at a time. Estrogen pellets are implanted subcutaneously for estrogen dependent tumors. After tumors from 5 mice come to size they are passaged into 24 NOG mice and tissue and plasma are collected with biological replicates at numerous time-points through the course of natural tumor progression in mice. The growth rates for all mice are known. To assess the effect of tumor size, mice undergo a complete, 75%, 50% or 25% debulking surgery when tumors reach ˜1.5 cm³ in size. Tumors harvested by resection are snap-frozen. Sham surgeries and no surgery serve as controls for tumor-bearing mice. There are 4 animals in each of these 6 groups totaling 24 animals per model to be tested. Blood is also collected by cheek bleeds prior to implantation, when tumors reach a palpable mass (˜150 mm³), biweekly until animals reach 1.5 cm³, after surgery and biweekly thereafter until mice become moribund, reach tumor volumes of 3 cm³, or after 20 weeks. For feasibility testing, Applicant has already performed debulking surgeries in a series of 3 models. Applicant has also determined that weekly cheek bleeds are tasking on the animals especially after surgery in tumor bearing animals so the biweekly regimen is much easier for mice to handle. DNA is extracted from tissue and plasma and processed for CpG4C by bAmplicon-seq as described earlier.

Since the models Applicant is utilizing form metastases in vivo, Applicant also performs serial necropsy and look for evidence of micro and macro metastasis in bones, liver, lung, brain, lymph nodes when tumors reach 1.0 cm³. For microscopic analysis, mouse organs are harvested and formalin-fixed and paraffin embedded. Entire organs are cryosectioned (5 μm) and stained with H&E. All organs are examined for evidence of micrometastases under the direction of the staff pathologist. For macrometastases, nodules are either visible in organs and/or animals arecome symptomatic (FIG. 12).

The CpG4C test are applied to each plasma sample to determine the timing of the first positive test, and whether the test remains positive after surgery (complete, or different degrees of debulking). Additionally, DNA methylation of individual CpGs are modeled as a function of time to determine the timing of methylation changes during disease progression and after treatment. Applicant uses flexible regression models (e.g. broken-line regression, or cubic splines) to identify at what point during disease progression DNA methylation changes occur, and whether certain CpG sites appear as earlier indicators of disease than others. Power considerations: Without being bound by theory, Applicant believes that the percent of positive tests are independent of tumor burden to the extent that this test is sensitive enough in a low tumor burden state. Assuming the test still achieves 50% sensitivity in the low tumor burden state, subjecting four mice to each treatment has 88% power to detect a positive test (5% significance level). This experiment are performed in 5 CpG4C positive PDX models of differing MBC subtype (e.g. Her2+, ER+, triple negative breast cancers) for either (1) verification that the loci that are most sensitive to residual disease are the same in different subtypes, or (2) to identify the most sensitive loci from a variety of disease subtypes.

### CpG4C Methylation Panel is Prognostic for Disease Recurrence in Early Stage Breast Cancer Patients.

The next step is to clinically validate if the CpG4C methylation panel can be detected in early stage breast cancer and if a positive CpG4C test can serve as a prognostic marker of recurrence. Since there is data on using cfDNA methylation for early detection of cancer and response to therapy in pre-metastatic settings, without being bound by theory, Applicant believes there is strong rationale to propose that CpG4C can detect cfDNA methylation in early stage breast cancer patients. In addition, data from Applicant's lab showing the detection of cfDNA in healthy and DFS samples along with low background signals of the target regions (FIG. 10) suggests this approach is possible.

Therefore, to clinically validate CpG4C, a study designed to collect blood before and after surgery in 100 consenting clinically high-risk patients who undergo neoadjuvant systemic therapy is performed (FIG. 14). By definition, patients who are candidates for a neoadjuvant treatment approach are considered high-risk. All patients have at least a Tlc tumor but no stage IV patients are recruited. The first blood sample are obtained at completion of neoadjuvant therapy before surgery. The second blood sample are obtained in the post-operative period (between 3-6 weeks. Patients are followed for recurrence by the medical oncologist as per standard of care and have additional therapy or imaging as the treating physician will see fit or as is directed by the patient's symptoms. A third and final blood draw and additional tissue are collected from patients with a recurrence. Blood are collected in a 10 ml EDTA lavender cap tube and processed for plasma according to SOPs in the lab as described in This example. Each tube yields ˜5 mls of plasma, which are cryopreserved in until further testing.

### Clinically Validate Whether CpG4C Can be a Prognostic Marker of Breast Cancer Recurrence

All study patients have blood samples drawn pre- and post-surgery. CpG4C test are performed on samples from both time points, and evaluated as a prognostic marker for disease recurrence. The CpG4C blood test pre-surgery will assess whether detection of microscopic residual disease following neoadjuvant therapy is prognostic of recurrence and therefore a better indicator of pCR. The second CpG4C blood test will assess the value of detecting microscopic residual disease to prognosticate recurrence after surgery. The results of the third blood sample, if taken, are compared to samples 1 and 2.

To ensure the highest reproducibility and avoid technical variability potentially associated with plasma extractions, two different lab personnel extract two independent aliquots. Cell-free DNA extractions are performed and quantitated as described earlier but in high-throughput (96-well format) and samples are randomized across plates. DNA goes through library preparation, bAmplicon-seq and bioinformatics analysis as described earlier.

Applicant classifies patients as positive or negative for CpG4C at the end of neo-adjuvant therapy and additionally at the post-operative blood draw based on cut-off criteria defined in This example. Disease-free survival (DFS) are compared between the two groups using Kaplan-Meier techniques with day 0 equal to the day of surgery or, in a separate analysis, at the day of the post-operative blood draw. A secondary analysis looks at the post-operative CpG4C result as a time-dependent covariate in a Cox Proportional Hazards model with recurrence as the response variable. Temporal patterns of CpG4C versus recurrence over time are also be assessed in a descriptive manner. Power is computed assuming a uniform recruitment rate over 3 years, with patients followed until recurrence or end of study. For a total of 100 patients, it is estimated that there are 33 CpG4C positive and 67 CpG4C negative patients. Further, it is estimated that 85% overall DFS at 5 years, 70% DFS in the CpG4C positive group and 92.5% DFS in the CpG4C negative group. With a one-sided alpha-level of 0.05, this study results in over 88% power to detect the above difference in DFS between patients with positive vs. negative CpG4C. The estimated power is still 83% if follow-up ends 1 year before end of study.

### Equivalents

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

### REFERENCES

1. Weigelt B, Peterse J L. Breast cancer metastasis: markers and models. Nature reviews Cancer. 2005; 5(8):591-602. doi:10.1038/nrc1670.
2. Blanco M A, Kang Y. Signaling pathways in breast cancer metastasis—novel insights from functional genomics. Breast cancer research: BCR. 2011; 13(2):206. doi:10.1186/bcr2831.
3. Chaffer C L, Weinberg R A. A perspective on cancer cell metastasis. Science. 2011; 331(6024):1559-64. doi:10.1126/science.1203543.
4. Mel nikov A A, Scholtens D, Talamonti M S, Bentrem D J, Levenson V V. Methylation profile of circulating plasma DNA in patients with pancreatic cancer. Journal of surgical oncology. 2009; 99(2):119-22. doi:10.1002/jso.21208.
5. Nakayama G, Hibi K, Nakayama H, Kodera Y, Ito K, Akiyama S, et al. A highly sensitive method for the detection of p16 methylation in the serum of colorectal cancer patients. Anticancer Res. 2007; 27(3B):1459-63.
6. Bastian P J, Palapattu G S, Yegnasubramanian S, Rogers C G, Lin X, Mangold L A, et al. CpG island hypermethylation profile in the serum of men with clinically localized and hormone refractory metastatic prostate cancer. J Urol. 2008; 179(2):529-34. doi:10.1016/j.juro.2007.09.038. discussion 34-5.
7. Fackler M J, Lopez Bujanda Z, Umbricht C, Teo W W, Cho S, Zhang Z, et al. Novel methylated biomarkers and a robust assay to detect circulating tumor DNA in metastatic breast cancer. Cancer research. 2014; 74(8):2160-70. doi:10.1158/0008-5472.CAN-13-3392.
8. Korshunova Y, Maloney R K, Lakey N, Citek R W, Bacher B, Budiman A, et al. Massively parallel bisulphite pyrosequencing reveals the molecular complexity of breast cancer-associated cytosine-methylation patterns obtained from tissue and serum DNA. Genome research. 2008; 18(1):19-29. doi:10.1101/gr.6883307.
9. Müller H M W A, Fiegl H, Ivarsson L, Goebe G, Perkmann E, Marth C, et al. DNA methylation in serum of breast cancer patients: an independent prognostic marker. Cancer research. 2003; 63(22):7641-5.
10. Chan K C, Jiang P, Chan C W, Sun K, Wong J, Hui E P, et al. Noninvasive detection of cancer-associated genome-wide hypomethylation and copy number aberrations by plasma DNA bisulfite sequencing. Proceedings of the National Academy of Sciences of the United States of America. 2013; 110(47):18761-8. doi:10.1073/pnas.1313995110.
11. Lau Q C, Raja E, Salto-Tellez M, Liu Q, Ito K, Inoue M, et al. RUNX3 is frequently inactivated by dual mechanisms of protein mislocalization and promoter hypermethylation in breast cancer. Cancer research. 2006; 66(13):6512-20. doi:10.1158/0008-5472.CAN-06-0369.
12. Miyamoto K, Fukutomi T, Akashi-Tanaka S, Hasegawa T, Asahara T, Sugimura T, et al. Identification of 20 genes aberrantly methyated in human breast cancers. International journal of cancer Journal international du cancer. 2005; 116(3):407-14. doi:10.1002/ijc.21054.
13. Kornegoor R, Moelans C B, Verschuur-Maes A H, Hogenes M, de Bruin P C, Oudejans J J, et al. Promoter hypermethylation in male breast cancer: analysis by multiplex ligation-dependent probe amplification. Breast cancer research: BCR. 2012; 14(4):R101. doi:10.1186/bcr3220.
14. Salhia B, Kiefer J, Ross J T, Metapaly R, Martinez R A, Johnson K N, et al. Integrated genomic and epigenomic analysis of breast cancer brain metastasis. PloS one. 2014; 9(1):e85448. doi:10.1371/journal.pone.0085448.
15. Appolloni I, Barilari M, Caviglia S, Gambini E, Reisoli E, Malatesta P. A cadherin switch underlies malignancy in high-grade gliomas. Oncogene. 2014. doi:10.1038/onc.2014.122.
16. Chung J H, Lee H J, Kim B H, Cho N Y, Kang G H. DNA methylation profile during multistage progression of pulmonary adenocarcinomas. Virchows Archiv: an international journal of pathology. 2011; 459(2):201-11. doi:10.1007/s00428-011-1079-9.
17. Xue T C, Ge N L, Zhang L, Cui J F, Chen R X, You Y, et al. Goosecoid promotes the metastasis of hepatocellular carcinoma by modulating the epithelial-mesenchymal transition. PloS one. 2014; 9(10):e109695. doi:10.1371/journal.pone.0109695.
18. Zhou L, Zhao X, Han Y, Lu Y, Shang Y, Liu C, et al. Regulation of UHRF1 by miR-146a/b modulates gastric cancer invasion and metastasis. FASEB journal: official publication of the Federation of American Societies for Experimental Biology. 2013; 27(12):4929-39. doi:10.1096/fj.13-233387.
19. Jao T M, Tsai M R, Lio H Y, Weng W T, Chen C C, Tzeng S T, et al. Protocadherin 10 suppresses tumorigenesis and metastasis in colorectal cancer and its genetic loss predicts adverse prognosis. International journal of cancer Journal international du cancer. 2014; 135(11):2593-603. doi:10.1002/ijc.28899.
20. Fackler M J, Umbricht C B, Williams D, Argani P, Cruz L A, Merino V F, et al. Genome-wide methylation analysis identifies genes specific to breast cancer hormone receptor status and risk of recurrence. Cancer research. 2011; 71(19):6195-207. doi:10.1158/0008-5472.CAN-11-1630.
21. Dawson S J, Tsui D W, Murtaza M, Biggs H, Rueda O M, Chin S F, et al. Analysis of circulating tumor DNA to monitor metastatic breast cancer. N Engl J Med. 2013; 368(13):1199-209. doi:10.1056/NEJMoa1213261.
22. Docherty S J, Davis O S, Haworth C M, Plomin R, Mill J. Bisulfite-based epityping on pooled genomic DNA provides an accurate estimate of average group DNA methylation. Epigenetics & chromatin. 2009; 2(1):3. doi:10.1186/1756-8935-2-3.
23. Kaplow I M, MacIsaac J L, Mah S M, McEwen L M, Kobor M S, Fraser H B. A pooling-based approach to mapping genetic variants associated with DNA methylation. Genome research. 2015; 25(6):907-17. doi:10.1101/gr.183749.114.
24. Gormally E, Caboux E, Vineis P, Hainaut P. Circulating free DNA in plasma or serum as biomarker of carcinogenesis: practical aspects and biological significance. Mutat Res. 2007; 635(2-3):105-17. doi:10.1016/j.mrrev. 2006.11.002.
25. Gormally E, Hainaut P, Caboux E, Airoldi L, Autrup H, Malaveille C, et al. Amount of DNA in plasma and cancer risk: a prospective study. International journal of cancer Journal international du cancer. 2004; 111(5):746-9. doi:10.1002/ijc.20327.
26. Bryzgunova O L P, Skvortsova T, Bondar A, Morozkin E, Lebedeva A, Krause H, et al. Efficacy of bisulfite modification and recovery of human genomic and circulating DNA using commercial kits. European Journal of Molecular Biology. 2013; 1(1):1-8. doi:10.11648/j.ejmb.20130101.11.
27. Byun H M, Nordio F, Coull B A, Tarantini L, Hou L, Bonzini M, et al. Temporal stability of epigenetic markers: sequence characteristics and predictors of short-term DNA methylation variations. PloS one. 2012; 7(6):e39220. doi:10.1371/journal.pone.0039220.
28. Krueger F, Andrews S R. Bismark: a flexibe aligner and methylation caller for Bisufite-Seq applications. Bioinformatics. 2011; 27(11):1571-2. doi:10.1093/bioinformatics/btr167.
29. Akalin A, Kormaksson M, Li S, Garrett-Bakelman F E, Figueroa M E, Melnick A, et al. methylKit: a comprehensive R package for the analysis of genome-wide DNA methylation profiles. Genome Biol. 2012; 13(10):R87. doi:10.1186/gb-2012-13-10-r87.

1. A method for detecting the level of DNA methylation in a sample isolated from a subject suspected of having or developing cancer or early stage cancer, the method comprising determining the level of DNA methylation at a genomic region within 10³ kb of at least one gene selected from RRAGC, RNF207, CAMTA1, IL17RE, Gp5, COX7B2, BANK1, LIMCH1, ANKRD33B, loc648987, HOXA2, NR_027387, ATG9B, KBTBD2, loc401321, MAFA, ANK1, SPAG1, PBX3, c9orf139, FUBP3, RABL6, DIP2C, CHFR, ZNF605, DZIP1, SLC35F4, ACSF2, ARHGAP23, FUZ, PBX4, UNC13A, ISM1, BMP2, loc286647, STAC2, TBX15, ESPN, PLEKHO1, c1orf95, HIVEp3, SPEG8, NR_038487, TANK, ARHGEF4, ZNF148, MIR548G, COX7B2, loc285548, Pi42Kb, PCDH7, FHDC1, GPR150, SLC6A3, VGLL2, NRN1, BLACE, WDR86, HOXA9, SOX17, ASS1, ALOX5, ZNF503, MAP6, EPS8L2, B4GALANT1, CLDN10, CLEC14A, PIF1, JPH3, SALL1, HIC1, ATP1B2, SRCIN1, NETO1, RCN3, and SEPT5-GP1BB in the sample.

2. (canceled)

3. A method for determining whether a subject is likely to have or develop cancer or cancer recurrence, the method comprising:
(a) determining the level of DNA methylation at a genomic region within 10³ kb of at least one gene selected from RRAGC, RNF207, CAMTA1, IL17RE, Gp5, COX7B2, BANK1, LIMCH1, ANKRD33B, loc648987, HOXA2, NR_027387, ATG9B, KBTBD2, loc401321, MAFA, ANK1, SPAG1, PBX3, c9orf139, FUBP3, RABL6, DIP2C, CHFR, ZNF605, DZIP1, SLC35F4, ACSF2, ARHGAP23, FUZ, PBX4, UNC13A, ISM1, BMP2, loc286647, STAC2, TBX15, ESPN, PLEKHO1, c1orf95, HIVEp3, SPEG8, NR_038487, TANK, ARHGEF4, ZNF148, MIR548G, COX7B2, loc285548, Pi42Kb, PCDH7, FHDC1, GPR150, SLC6A3, VGLL2, NRN1, BLACE, WDR86, HOXA9, SOX17, ASS1, ALOX5, ZNF503, MAP6, EPS8L2, B4GALANT1, CLDN10, CLEC14A, PIF1, JPH3, SALL1, HIC1, ATP1B2, SRCIN1, NETO1, RCN3, and SEPT5-GP1BB in a sample isolated from the subject;
(b) comparing the level of DNA methylation in the sample to the level of DNA methylation in a sample isolated from a cancer-free subject, a normal reference standard, or a normal reference cutoff value;
(c) determining that the subject is likely to have or develop cancer or cancer recurrence if the level of DNA methylation in the sample derived from the subject is greater than the level of DNA methylation in the sample isolated from a cancer-free subject, a normal reference standard, or a normal reference cutoff value.

4. A method for detecting the level of DNA methylation in a sample isolated from a subject suspected of having or developing cancer or early stage cancer, the method comprising determining the level of DNA methylation at a genomic region within 10³ kb of at least one gene selected from RRAGC, RNF207, CAMTA1, IL17RE, Gp5, COX7B2, BANK1, LIMCH1, ANKRD33B, loc648987, HOXA2, NR_027387, ATG9B, KBTBD2, loc401321, MAFA, ANK1, SPAG1, PBX3, c9orf139, FUBP3, RABL6, DIP2C, CHFR, ZNF605, DZIP1, SLC35F4, ACSF2, ARHGAP23, FUZ, PBX4, UNC13A, ISM1, BMP2, loc286647, STAC2, TBX15, ESPN, PLEKHO1, c1orf95, HIVEp3, SPEG8, NR_038487, TANK, ARHGEF4, ZNF148, MIR548G, COX7B2, loc285548, Pi42Kb, PCDH7, FHDC1, GPR150, SLC6A3, VGLL2, NRN1, BLACE, WDR86, HOXA9, SOX17, ASS1, ALOX5, ZNF503, MAP6, EPS8L2, B4GALANT1, CLDN10, CLEC14A, PIF1, JPH3, SALL1, HIC1, ATP1B2, SRCIN1, NETO1, RCN3, and SEPT5-GP1BB in the sample.

5-34. (canceled)

35. The method of claim 1, wherein the level of DNA methylation is determined at one or more CpG islands within 10³ kb of the selected gene or genes.

36. (canceled)

37. The method of claim 1, wherein the level of DNA methylation is determined at a genomic region within the selected gene or genes.

38. The method of claim 37, wherein the level of DNA methylation is determined at a genomic region within an untranslated region (UTR) of the selected gene or genes.

39. The method of claim 37, wherein the level of DNA methylation is determined at a genomic region within 1.5 kb upstream of the transcription start site of the selected gene or genes.

40. The method of claim 37, wherein the level of DNA methylation is determined at a genomic region within the first exon of the selected gene or genes.

41. A method for determining whether a subject is likely to have or develop cancer or early stage cancer, the method comprising:
(a) determining the level of DNA methylation at one or more genomic regions selected from chr1:119,522,297-119,522,685, chr1:150,122,865-150,123,881, chr1:226,736,415-226,736,530, chrl_:228,651,389-228,652,669, chrl_:39,044,074-39,044,222, chrl_:39,044,074-39,044,225, chr1:39,269,706-39,269,850, chr1:42,383,685-42,383,856, chr1:6,268,888-6,269,045, chr1:6,508,634-6,508,912, chr1:7,765,055-7,765,179, chr2:131,792,795-131,792,937, chr2:162,100,925-162,101,769, chr2:208,989,125-208,989,413, chr2:220,313,284-220,313,454, chr2:220,313,294-220,313,436, chr2:468,028-468,289, chr3:125,076,002-125,076,434, chr3:194,117,552-194,119,057, chr3:194,117,921-194,118,045, chr3:9,957,033-9,957,468, chr3:99,595,058-99,595,326, chr4:102,712,059-102,712,200, chr4:13,549,015-13,549,160, chr4:153,858,813-153,858,916, chr4:25,235,927-25,236,058, chr4:30,723,649-30,723,941, chr4:41,646,367-41,646,493, chr4:46,726,419-46,726,619, chr4:46,726,427-46,726,601, chr4:46,726,525-46,726,603, chr4:8,895,441-8,895,846, chr5:1,445,269-1,445,490, chr5:10,565,517-10,565,682, chr5:43,018,031-43,018,972, chr5:94,955,846-94,956,706, chr6:117,591,888-117,592,164, chr6:6,002,430-6,002,857, chr7:150,715,883-150,715,989, chr7:151,106,717-151,106,910, chr7:152,161,438-152,161,508, chr7:155,167,043-155,167,243, chr7:27,141,743-27,141,932, chr7:27,204,874-27,205,029, chr7:32,801,782-32,802,525, chr7:32,930,792-32,930,842, chr8:101,225,311-101,225,367, chr8:144,511,850-144,512,138, chr8:41,655,108-41,655,453, chr8:55,379,115-55,379,416, chr9:128,510,274-128,510,341, chr9:133,308,833-133,309,057, chr9:133,454,823-133,454,962, chr9:139,715,901-139,716,003, chr9:139,925,051-139,925,313, chr10:45,914,402-45,914,709, chr10:735,378-735,552, chr10:77,156,043-77,156,222, chr11:725,576-725,843, chr11:75,379,637-75,379,770, chr12:133,481,446-133,481,616, chr12:58,021,185-58,021,918, chr13:29,393,957-29,394,126, chr13:96,204,915-96,205,232, chr13:96,293,984-96,294,377, chr14:38,724,432-38,725,600, chr14:58,332,639-58,332,759, chr15:65,116,372-65,116,575, chr15:66,914,674-66,914,722, chr16:51,185,202-51,185,325, chr16:87,636,189-87,636,318, chr17:1,960,496-1,960,610, chr17:36,666,487-36,666,582, chr17:36,714,476-36,714,611, chr17:37,366,246-37,366,533, chr17:37,381,269-37,381,871, chr17:44,337,407-44,337,726, chr17:48,546,161-48,546,934, chr17:7,554,926-7,555,051, chr18:70,522,481-70,548,676, chr19:17,716,756-17,717,092, chr19:19,729,144-19,729,553, chr19:30,716,841-30,717,033, chr19:50,030,948-50,031,354, chr19:50,312,537-50,312,694, chr20:13,200,413-13,200,789, chr20:6,748,289-6,748,421, chr22:19,711,302-19,711,474, and chrX:130,929,860-130,930,244 in a sample isolated from the subject;
(b) comparing the level of DNA methylation in the sample to the level of DNA methylation in a sample isolated from a cancer-free subject, a normal reference standard, or a normal reference cutoff value;
(c) determining that the subject is likely to have or develop cancer or cancer recurrence if the level of DNA methylation in the sample derived from the subject is greater than the level of DNA methylation in the sample isolated from a cancer-free subject, a normal reference standard, or a normal reference cutoff value.

42.-72. (canceled)

73. The method of claim 1, wherein the DNA methylation level is determined with targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

74. (canceled)

75. The method of claim 1, further comprising one or more of targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot.

76.-84. (canceled)

85. The method of claim 1, wherein the cancer is lung cancer, breast cancer, colorectal cancer, prostate cancer, stomach cancer, liver cancer, cervical cancer, esophageal cancer, bladder cancer, non-Hodgkin lymphoma, leukemia, pancreatic cancer, kidney cancer, endometrial cancer, oral cancer, thyroid cancer, brain cancer, nervous system cancer, ovarian cancer, uterine cancer, melanoma, gallbladder cancer, laryngeal cancer, multiple myeloma, nasopharyngeal cancer, Hodgkin lymphoma, testicular cancer, or Kaposi sarcoma.

86.-89. (canceled)
